(19) United States
(12) Patent Application Publication
(10) Pub. No.: US 2018/0279965 A1
(43) Pub. Date: Oct. 4, 2018

(54) Ambulatory Blood Pressure and Vital Sign Monitoring Apparatus, System and Method

(71) Applicant: Northwestern University, Evanston, IL (US)

(72) Inventors: Jay A. Pandit, Elmhurst, IL (US); Kyle R. Miller, San Jose, CA (US); Sean D. Connell, Houston, TX (US); Jung-en Wu, New Taipei City (TW)

(21) Appl. No.: 15/764,538

(22) PCT Filed: Oct. 11, 2016
(86) PCT No.: PCT/US16/56350

§ 371 Date: Mar. 29, 2018

Related U.S. Application Data
(60) Provisional application No. 62/240,360, filed on Oct. 12, 2015.
(60) Provisional application No. 62/343,256, filed on May 31, 2016.

(51) Int. Cl. A61B 5/00; A61B 5/021; A61B 5/02; A61B 5/01; A61B 5/11

(52) U.S. Cl. CPC A61B 5/7278; A61B 2562/0238; A61B 5/02028; A61B 5/7264; A61B 5/7225; A61B 5/0015; A61B 5/01; A61B 5/11; A61B 5/742; A61B 5/6806; A61B 5/681; A61B 2560/0223; A61B 2562/0247; A61B 2562/0219; A61B 5/02125

(57) ABSTRACT

Representative methods, apparatus and systems are disclosed for determining one or more physiological parameters, such as for ambulatory blood pressure and other vital sign monitoring. A representative system comprises first and second wearable apparatuses to be worn on the user's left and right sides, and any of several types of central vital signs monitors. Another representative system is a handheld, singular apparatus to be held in both hands by the user. Another representative system comprises first and second wearable apparatuses without any additional central vital signs monitor. The various embodiments measure a differential pulse arrival time of left and right arterial pressure waves using corresponding determined features, such as a foot or systolic peak, and using the measured differential pulse arrival time and calibration data, determine at least one physiological parameter such as blood pressure, heart rate, stroke rate, and cardiac output.

### CROSS-REFERENCE TO A RELATED APPLICATION

This application is a U.S. national phase under 35 U.S.C. Section 371 and claims the benefit of and priority to International Application No. PCT/US2016/056350 filed Oct. 11, 2016, which is a nonprovisional of and claims the benefit of and priority to U.S. Provisional Patent Application No. 62/343,256, filed May 31, 2016, inventors Jung-En Wu et al., titled “Ambulatory Blood Pressure and Vital Sign Monitoring Apparatus, System and Method”, and further is a nonprovisional of and claims the benefit of and priority to U.S. Provisional Patent Application No. 62/240,360, filed Oct. 12, 2015, inventors Jung-En Wu et al., titled “Ambulatory Blood Pressure Monitor”, which are commonly assigned herewith, and all of which are hereby incorporated herein by reference in their entireties with the same full force and effect as if set forth in their entireties herein.

### FIELD OF THE INVENTION

The present invention, in general, relates to blood pressure and other vital sign monitoring, and more particularly, relates to an apparatus, system and method for noninvasive, ambulatory blood pressure and vital sign monitoring.

### BACKGROUND OF THE INVENTION

High blood pressure (“BP”), also referred to as hypertension, is a major cardiovascular risk factor contributing to various medical conditions, diseases, and events such as heart attacks, heart failure, aneurisms, strokes, and kidney disease, for example. While hypertension generally is medically treatable, the rates for detection and control of high BP remain low, especially because high BP may not cause any other symptoms which would be noticeable to an individual. As a result, there is a well-established need for blood pressure and other vital sign monitoring, whether such monitoring occurs in a hospital setting, a physician's office, a patient's home or office, and whether such monitoring occurs while the individual is at rest or engaged in an activity, such as sitting, walking, exercising, or sleeping, also for example.

For a wide variety of reasons, there is also a growing need for ubiquitous, continuous, and/or ambulatory BP monitoring, which may be conducted by an individual away from a hospital, clinic or physician's office. For example, BP monitoring may be necessary for determining whether the individual has hypertension in fact, or simply has high BP in a clinical setting and does not require medical treatment (a condition often referred to as “white coat hypertension”). BP monitoring may be necessary for determining the response to and proper dosages of blood pressure medications prescribed for an individual. BP monitoring also may be necessary for determining the times of day and types of activity of an individual which tend to raise or lower the individual's blood pressure, such as whether an individual's BP is lower while sleeping or reading, or higher when drinking coffee, driving, or attending work meetings, for example.

Existing methods of determining BP have limited applicability to blood pressure and other vital sign monitoring in many of these settings. For example, BP monitoring technologies using catheterization are highly invasive and may only be performed in hospital or other clinical settings. Other technologies, such as auscultation or oscillometry, typically utilize a pressurized cuff to occlude an artery, which is followed during cuff deflation by detection of Korotkoff sounds using a stethoscope in conjunction with pressure determinations, typically using a manometer or a pressure sensor inside the cuff. While generally accurate under many circumstances, these cuffs are cumbersome, inconvenient, time consuming to use, and are disruptive during ambulatory monitoring, especially during sleep. Pressurized cuff methodologies are also unsuitable for certain environments, such as at high altitude, at the higher levels of the atmosphere, and in space. These methods and apparatus are also comparatively expensive, limiting their utility in certain settings, such as in low resource settings.

Another, cuffless methodology has attempted to utilize pulse transit time (“PTT”) as a BP indicator for ambulatory BP monitoring. PTT, which is the time delay for a pulse pressure wave to travel between two arterial sites, has an inverse relationship with BP, with a higher BP resulting in a lower PTT. Existing PTT methodologies suffer from several problems, however, including difficulties in measuring the PTT, difficulties in calibrating an individual's PTT with the individual's BP, along with significant inaccuracy due to various factors, such as interference from noise and user movement, along with effectively false or inaccurate BP determinations due to changes in measured PTT due from hydrostatic and hydrodynamic factors without actual corresponding changes in the arterial BP in the vicinity of the heart.

Accordingly, there is an ongoing need for a new apparatus, method and/or system for noninvasive, ambulatory blood pressure and other vital sign monitoring. Such an apparatus and/or system should be comparatively unobtrusive, convenient and easy to use for an individual consumer, while nonetheless being comparatively or sufficiently accurate to obtain meaningful results and actionable information, with a comparatively fast BP acquisition time. Such an apparatus, method and/or system should provide improved compliance by being readily integrable into the user's daily activities. Depending on the selected embodiment, such a technology should be readily portable and/or wearable to provide ubiquitous monitoring all day and/or night, as may be necessary or desirable.

### SUMMARY OF THE INVENTION

As discussed in greater detail below, the representative apparatus, system and method provide for determining a physiological parameter of a subject human being for monitoring, such as a noninvasive, ambulatory blood pressure and other vital sign monitoring. A representative physiological parameter monitoring apparatus, method and system, such as for BP and other vital sign monitoring, utilize measurements of a differential pulse arrival time (“DPAT”), also discussed in greater detail below, as an indicator of BP, which are obtained at symmetrical left and right locations along human peripheral arteries, such as at generally symmetrical left and right locations or positions on an individual's ears, neck, upper or lower arms, wrists, fingers, or fingertips. Other vital signs, as physiological parameters, may also be determined, including without limitation heart rate, cardiac output, stroke volume, and oxygen saturation.

The representative embodiments of the present invention provide numerous advantages. The representative apparatus, method and/or system embodiments provide for determining a physiological parameter of a subject human being for monitoring, such as noninvasive, ambulatory blood pressure and other vital sign monitoring. Representative apparatus and/or system embodiments are comparatively unobtrusive, convenient and easy to use for an individual consumer, while nonetheless being comparatively or sufficiently accurate to obtain meaningful results and actionable information, with a comparatively fast BP acquisition time. Representative apparatus and/or system embodiments also may provide improved compliance by being readily integrable into the user's daily activities. Depending on the selected embodiment, such representative apparatus and/or system embodiments are readily portable and/or wearable to provide ubiquitous monitoring all day and/or night, as may be necessary or desirable.

A representative method embodiment for determining a physiological parameter of a subject human being for monitoring is disclosed, the subject having a left side and a right side, with the representative method comprising: generating a left signal and a right signal to corresponding left and right positions on the subject; receiving left and right analog sensor electrical signals from corresponding left and right positions on the subject; sampling and converting the left and right analog sensor electrical signals into a plurality of digital amplitude values representing amplitudes of left and right arterial pressure waves; determining corresponding features of the left and right arterial pressure waves; using the corresponding determined features, measuring a differential pulse arrival time of the left and right arterial pressure waves; and using the measured differential pulse arrival time, determining at least one physiological parameter selected from the group consisting of: blood pressure, heart rate, stroke rate, and cardiac output.

For example, the corresponding left and right positions on the subject comprise the subject's neck, ears, and upper extremities, such as arms, wrists, fingers, and fingertips.

In a representative embodiment, when the determined physiological parameter is to be blood pressure, the step of determining at least one physiological parameter further comprises: using calibration data for the subject, mapping the measured differential pulse arrival time to a corresponding blood pressure determined by the calibration data. For example, for any of the various embodiments, the mapping may be selected from the group consisting of: a nonlinear, sigmoidal mapping; a piece-wise linear mapping; a nonlinear autoregressive exogenous mapping; an artificial neural network mapping; a recursive Bayesian network mapping; and combinations thereof.

Also for example, for any of the various embodiments, the calibration data may comprise a plurality of differential pulse arrival times determined for a corresponding plurality of independently determined blood pressure values. As another example, the calibration data may comprise a plurality of differential pulse arrival times determined for a corresponding plurality of independently determined blood pressure values, a plurality of movements, a plurality of temperatures, and a plurality of sensor pressures.

In a representative embodiment, the method may also include generating a plurality of first derivatives of the plurality of digital amplitude values. In a representative embodiment, the corresponding determined features may be a corresponding foot of the left and right arterial pressure waves, determined using the plurality of first derivatives, the plurality of first derivatives indicating a diastolic minimum before a systolic peak and indicating a maximum rate of increasing change in the pressure wave at a rising edge of the systolic peak.

In a representative embodiment, for example, the generated left and right signals are optical signals in a predetermined wavelength band.

A representative method may further comprise: using a temperature sensor, receiving temperature data; and using a pressure sensor, receiving pressure data. For such an embodiment, when the determined physiological parameter is blood pressure, the representative method may further comprise modifying the determined blood pressure based upon the received temperature and pressure data. A representative method may further comprise: using an accelerometer, receiving movement data; and modifying the determined blood pressure based upon the received movement data. A representative method also may further comprise filtering the plurality of digital amplitude values.

A representative method may further comprise: displaying the determined physiological parameter value, such as a blood pressure value and other vital sign information, to the user; and/or transmitting the determined physiological parameter value, such as a blood pressure value and other vital sign information, to a central location; and/or storing the determined physiological parameter value, such as a blood pressure value and other vital sign information, in a memory circuit.

A system for determining a physiological parameter of a subject human being for monitoring is also disclosed, the subject having a left side and a right side, with a representative system comprising a plurality of wearable apparatuses and a central vital signs monitor. A first wearable apparatus is adapted to be worn on the left side, a second wearable apparatus is adapted to be worn on the right side, with each wearable apparatus of the plurality of wearable apparatuses comprising: a signal generator to generate either a left signal or a right signal to corresponding left and right positions on the subject; a sensor to receive a left or right analog sensor electrical signal from corresponding left and right positions on the subject; an analog-to-digital converter coupled to the sensor to sample and convert the left and right analog sensor electrical signals into a plurality of digital amplitude values representing amplitudes of left and right arterial pressure waves; and a wireless transmitter coupled to the analog-to-digital converter, the wireless transmitter to transmit the plurality of digital amplitude values. The central vital signs monitor comprises: a memory circuit to store calibration data for the subject; a wireless transceiver to receive the transmitted plurality of digital amplitude values; and a processor coupled to the wireless transceiver and to the memory, the processor adapted to determine corresponding features of the left and right arterial pressure waves; measure a differential pulse arrival time of the left and right arterial pressure waves using the corresponding determined features; and using the measured differential pulse arrival time and the calibration data, to determine at least one physiological parameter selected from the group consisting of: blood pressure, heart rate, stroke rate, and cardiac output.

Another representative system is disclosed for determining a physiological parameter of a subject human being for monitoring, the subject having a left side and a right side, with the representative system comprising a first wearable apparatus and a second wearable apparatus. The first wearable apparatus is adapted to be worn on the left or right sides, with the first wearable apparatus comprising: a first signal generator to generate either a left signal or a right signal to corresponding left or right positions on the subject; a first sensor to receive a left or right analog sensor electrical signal from corresponding left and right positions on the subject; a first analog-to-digital converter coupled to the first sensor to sample and convert the left or right analog sensor electrical signals into a first plurality of digital amplitude values representing amplitudes of left or right arterial pressure waves; and a wireless transmitter coupled to the first analog-to-digital converter, the wireless transmitter to transmit the plurality of digital amplitude values. The second wearable apparatus is adapted to be worn on the corresponding right or left side, with the second wearable apparatus comprising: a second signal generator to generate either a right signal or a left signal to corresponding right or left positions on the subject; a second sensor to receive a right or left analog sensor electrical signal from corresponding right or left positions on the subject; a second analog-to-digital converter coupled to the second sensor to sample and convert the right or left analog sensor electrical signals into a second plurality of digital amplitude values representing amplitudes of right or left arterial pressure waves; a memory circuit to store calibration data for the subject; a wireless transceiver to receive the transmitted first plurality of digital amplitude values; and a processor coupled to the wireless transceiver and to the memory, the processor adapted to determine corresponding features of the left and right arterial pressure waves; measure a differential pulse arrival time of the left and right arterial pressure waves using the corresponding determined features; and using the measured differential pulse arrival time and the calibration data, to determine at least one physiological parameter selected from the group consisting of: blood pressure, heart rate, stroke rate, and cardiac output.

A representative apparatus is also disclosed for determining a physiological parameter of a subject human being for monitoring, the subject having a left side and a right side, with the representative apparatus comprising: a housing having a first, left finger placement location and a second, right finger placement location; a first signal generator arranged within the housing at the first finger placement location to generate a left signal to a left finger of the subject; a second signal generator arranged within the housing at the second finger placement location to generate a right signal to a right finger of the subject; a first sensor arranged within the housing at the first finger placement location to receive a left analog sensor electrical signal from the left finger of the subject; a second sensor arranged within the housing at the second finger placement location to receive a right analog sensor electrical signal from a right finger of the subject; a first analog-to-digital converter arranged within the housing and coupled to the first sensor to sample and convert the left analog sensor electrical signals into a first plurality of digital amplitude values representing amplitudes of a left arterial pressure wave; a second analog-to-digital converter arranged within the housing and coupled to the second sensor to sample and convert the right analog sensor electrical signals into a second plurality of digital amplitude values representing amplitudes of a right arterial pressure wave; a memory circuit arranged within the housing to store calibration data for the subject; a processor arranged within the housing and coupled to the memory and to the first and second analog-to-digital converters, the processor adapted to determine corresponding features of the left and right arterial pressure waves; measure a differential pulse arrival time of the left and right arterial pressure waves using the corresponding determined features; and using the measured differential pulse arrival time and the calibration data, to determine at least one physiological parameter selected from the group consisting of: blood pressure, heart rate, stroke rate, and cardiac output.

Another representative apparatus is disclosed for determining a physiological parameter of a subject human being for monitoring, the subject having a left side and a right side, with the apparatus utilized in conjunction with a computing device, with the apparatus comprising: a housing having a first, left finger placement location and a second, right finger placement location; a first signal generator arranged within the housing at the first finger placement location to generate a left signal to a left finger of the subject; a second signal generator arranged within the housing at the second finger placement location to generate a right signal to a right finger of the subject; a first sensor arranged within the housing at the first finger placement location to receive a left analog sensor electrical signal from the left finger of the subject; a second sensor arranged within the housing at the second finger placement location to receive a right analog sensor electrical signal from a right finger of the subject; a first analog-to-digital converter arranged within the housing and coupled to the first sensor to sample and convert the left analog sensor electrical signals into a first plurality of digital amplitude values representing amplitudes of a left arterial pressure wave; a second analog-to-digital converter arranged within the housing and coupled to the second sensor to sample and convert the right analog sensor electrical signals into a second plurality of digital amplitude values representing amplitudes of a right arterial pressure wave; and a wireless transmitter coupled to the first and second analog-to-digital converters to transmit the first and second pluralities of digital amplitude values to the computing device.

For such a representative embodiment, the computing device comprises: a wireless transceiver to receive the first and second pluralities of digital amplitude values; a memory circuit to store calibration data for the subject; and a processor coupled to the memory and to the wireless transceiver, the processor adapted to determine corresponding features of the left and right arterial pressure waves; measure a differential pulse arrival time of the left and right arterial pressure waves using the corresponding determined features; and using the measured differential pulse arrival time and the calibration data, to determine at least one physiological parameter selected from the group consisting of: blood pressure, heart rate, stroke rate, and cardiac output.

In a representative embodiment, when the determined physiological parameter is blood pressure, the processor is further adapted to determine the blood pressure by mapping the measured differential pulse arrival time to a corresponding blood pressure determined by the calibration data, wherein the mapping is selected from the group consisting of: a nonlinear, sigmoidal mapping; a piece-wise linear mapping; a nonlinear autoregressive exogenous mapping; an artificial neural network mapping; a recursive Bayesian network mapping; and combinations thereof.

In a representative embodiment, the processor may be further adapted to generate a plurality of first derivatives of the plurality of digital amplitude values; and to determine a corresponding foot of the left and right arterial pressure waves as the corresponding determined features, using the plurality of first derivatives, the plurality of first derivatives indicating a minimum before a systolic peak and indicating a maximum rate of increasing change in the pressure wave at a rising edge of the systolic peak.

In a representative embodiment, the signal generator may be an optical signal generator to generate light in a predetermined wavelength band.

In a representative embodiment, each wearable apparatus may further comprise: a temperature sensor to receive temperature data; and a pressure sensor to receive pressure data; wherein the processor is further adapted to modify the determined blood pressure based upon the received temperature and pressure data.

In a representative embodiment, each wearable apparatus may further comprise: an accelerometer to receive movement data; wherein the processor is further adapted to modify the determined blood pressure based upon the received movement data. In another representative embodiment, for example, the processor is further adapted to filter the plurality of digital amplitude values.

For any of the various embodiments, either the central vital signs monitor or one of the wearable apparatus may further comprise: a visual display device to display the determined blood pressure value and other vital sign information to the user.

For any of the various embodiments, the wireless transceiver may be further adapted to transmit the determined blood pressure value and other vital sign information to a central location. Also for any of the various embodiments, the processor may be further adapted to store the determined blood pressure value and other vital sign information in the memory circuit.

In a representative embodiment, at least one of the wearable apparatus further comprises a wearable attachment selected from the group consisting of: an adhesive patch, a wristband, a finger ring, a finger sleeve, a finger clip, a glove, an ear clip, and a bracelet.

In another representative embodiment, the central vital signs monitor is embodied in a separate computing device.

Numerous other advantages and features of the present invention will become readily apparent from the following detailed description of the invention and the embodiments thereof, from the claims and from the accompanying drawings.

### BRIEF DESCRIPTION OF THE DRAWINGS

The objects, features and advantages of the present invention will be more readily appreciated upon reference to the following disclosure when considered in conjunction with the accompanying drawings, wherein like reference numerals are used to identify identical components in the various views, and wherein reference numerals with alphabetic characters are utilized to identify additional types, instantiations or variations of a selected component embodiment in the various views, in which:

FIG. 1 is a graphical diagram illustrating respective amplitudes over time of representative right and left arterial pressure waves, and a corresponding DPAT, obtained at symmetrical right and left locations or positions in the neck, ears or upper extremities of an individual.

FIG. 2 is a graphical diagram illustrating a plurality of digital samples of a representative arterial pressure wave obtained at a location or position in the neck, ear, or upper extremity of an individual and a BP waveform foot feature.

FIG. 3 is a graphical diagram illustrating a baseline differential pulse arrival time from representative right and left arterial pressure waves obtained at symmetrical right and left locations or positions in the neck, ears or upper extremities of an individual when the individual is at rest.

FIG. 4 is a graphical diagram illustrating an increased differential pulse arrival time from representative right and left arterial pressure waves obtained at symmetrical right and left locations or positions in the neck, ears or upper extremities of an individual, following performance of a Valsalva maneuver.

FIG. 5 is a graphical diagram illustrating a decreased differential pulse arrival time from representative right and left arterial pressure waves obtained at symmetrical right and left locations or positions in the neck, ears or upper extremities of an individual, following exercise.

FIG. 6 is a graphical diagram illustrating a decreased differential pulse arrival time from representative right and left arterial pressure waves obtained at symmetrical right and left locations or positions in the neck, ears or upper extremities of an individual, following a cold pressor test.

FIGS. 7A and 7B (collectively referred to as FIG. 7) are bar chart diagrams illustrating, in FIG. 7A, a baseline blood pressure and increased blood pressures of an individual at rest, and following a cold pressor test and following exercise, and in FIG. 7B, corresponding baseline and decreased differential pulse arrival times from representative right and left arterial pressure waves obtained at symmetrical right and left locations or positions in the neck, ears or upper extremities of the individual at rest, and following a cold pressor test and following exercise.

FIG. 8 is a graphical diagram illustrating an increased differential pulse arrival time from representative right and left arterial pressure waves obtained at symmetrical right and left locations or positions in the neck, ears or upper extremities of an individual, following performance of a Valsalva maneuver, over a sixty second period.

FIG. 9 is a graphical diagram illustrating a decreased differential pulse arrival time (less negative) from representative right and left arterial pressure waves obtained at symmetrical right and left locations or positions in the neck, ears or upper extremities of an individual, following exercise, over a sixty second period.

FIG. 10 is a graphical diagram illustrating a decreased differential pulse arrival time (less negative) from representative right and left arterial pressure waves obtained at symmetrical right and left locations or positions in the neck, ears or upper extremities of an individual, following a cold pressor test, over a sixty second period.

FIG. 11 is a block diagram of representative first apparatus and first system embodiments.

FIG. 12 is a block diagram of representative second apparatus and second system embodiments.

FIG. 13 is a block diagram of representative third apparatus and third system embodiments.

FIG. 14 is a block diagram of representative fourth apparatus and fourth system embodiments.

FIGS. 15A and 15B (collectively referred to as FIG. 15) is a flow chart of a representative method embodiment for the determination of systolic and diastolic blood pressure values, heart rate and other vital signs.

FIG. 16 is a flow chart of a representative method embodiment for the calibration of the representative apparatus and system embodiments for the determination of systolic and diastolic blood pressure values, heart rate and other vital signs.

FIGS. 17A and 17B (collectively referred to as FIG. 17) are graphical diagram illustrating, in FIG. 17A, collected DPAT measurements or determinations and mean arterial BP measurements performed and collected using an independent BP measuring device and in FIG. 17B, estimated systolic BP values from collected DPAT measurements or determinations, and systolic BP measurements performed and collected using the independent BP measuring device.

FIG. 18 is a graphical diagram illustrating estimated diastolic BP values from collected DPAT measurements or determinations, and diastolic BP measurements performed using the independent BP measuring device.

FIG. 19 is a graphical diagram illustrating collected DPAT measurements or determinations for systolic BP measurements or determinations, and systolic BP measurements performed using the independent BP measuring device, for calibration of DPAT measurements or determinations over first and second hydrostatic and/or hydrodynamic movements, conditions or events.

FIG. 20 is a graphical diagram illustrating collected DPAT measurements or determinations for systolic BP measurements or determinations, and systolic BP measurements performed using the independent BP measuring device, for calibration of DPAT measurements or determinations over third and fourth hydrostatic and/or hydrodynamic movements, conditions or events.

FIG. 21 is a graphical diagram of FIGS. 19 and 20 illustrating collected DPAT measurements or determinations for systolic BP measurements or determinations, and systolic BP measurements performed using the independent BP measuring device, for calibration of DPAT measurements or determinations over first, second, third and fourth hydrostatic and/or hydrodynamic movements, conditions or events, using a piece-wise linear calibration mapping.

FIG. 22 is a graphical diagram of FIGS. 19 and 20 illustrating collected DPAT measurements or determinations for systolic BP measurements or determinations, and systolic BP measurements performed using the independent BP measuring device, for calibration of DPAT measurements or determinations over first, second, third and fourth hydrostatic and/or hydrodynamic movements, conditions or events, using a nonlinear, sigmoidal calibration mapping.

FIG. 23 is an isometric view diagram illustrating representative first, second and/or third apparatus embodiments with a wearable wristband attachment.

FIG. 24 is an isometric view diagram illustrating representative first, second and/or third apparatus embodiments with a wearable ring attachment.

FIGS. 25A, 25B, 25C, 25D, 25E and 25F (collectively referred to as FIG. 25) are isometric view diagrams illustrating representative first, second and/or third apparatus embodiments with, in FIGS. 25A, 25B, 25C, and 25D, a wearable wristband attachment, in FIG. 25E, a wearable adhesive patch attachment, and in FIG. 25F, a representative first, second and/or third apparatus embodiment with a wearable wristband attachment attached around a wrist of a human subject.

FIG. 26 is an isometric view diagram illustrating representative first, second and/or third apparatus embodiment with a wearable wristband attachment attached around a wrist of a human subject.

FIG. 27 is an isometric view diagram illustrating representative first, second, third and/or fourth apparatus embodiments arranged within a housing such as a smartphone case.

FIG. 28 is an isometric, rear view diagram illustrating a representative fourth apparatus embodiment arranged within a housing.

FIG. 29 is an isometric, front view diagram illustrating a representative fourth apparatus embodiment arranged within a housing.

### DETAILED DESCRIPTION OF REPRESENTATIVE EMBODIMENTS

While the present invention is susceptible of embodiment in many different forms, there are shown in the drawings and will be described herein in detail specific exemplary embodiments thereof, with the understanding that the present disclosure is to be considered as an exemplification of the principles of the invention and is not intended to limit the invention to the specific embodiments illustrated. In this respect, before explaining at least one embodiment consistent with the present invention in detail, it is to be understood that the invention is not limited in its application to the details of construction and to the arrangements of components set forth above and below, illustrated in the drawings, or as described in the examples. Methods and apparatuses consistent with the present invention are capable of other embodiments and of being practiced and carried out in various ways. Also, it is to be understood that the phraseology and terminology employed herein, as well as the abstract included below, are for the purposes of description and should not be regarded as limiting.

As mentioned above and as discussed in greater detail below, the representative apparatus, system and method provide for determining a physiological parameter of a subject human being for monitoring, such as a noninvasive, ambulatory blood pressure and other vital sign monitoring. A representative apparatus, system and method will determine at least one physiological parameter such as blood pressure, heart rate, stroke rate, and cardiac output.

For ease of explanation, the various representative embodiments are discussed in greater detail below with reference to determinations of a subject individual's blood pressure, as a highly useful and valuable example of a physiological parameter. Those having skill in the art will recognize that the various representative embodiments also more broadly provide for determination of a wide variety of physiological parameters in addition to blood pressure, such as heart rate, stroke rate, and cardiac output. Accordingly, the representative apparatus, system and method should not be regarded, in any way, as limited to blood pressure monitoring, and all such representative embodiments should be understood to mean and include the capabilities for determining at least one physiological parameter such as blood pressure, heart rate, stroke rate, and cardiac output.

A representative physiological parameter monitoring apparatus, method and system, such as for BP and other vital sign monitoring, utilize measurements or other determinations of a differential pulse arrival time, also discussed in greater detail below, as an indicator of BP, which are obtained at symmetrical left and right locations along human peripheral arteries, such as at generally symmetrical left and right locations or positions on an individual's ears, neck, upper or lower arms, wrists, fingers, or fingertips. Other vital signs may also be determined, including without limitation heart rate, cardiac output, stroke volume, and oxygen saturation.

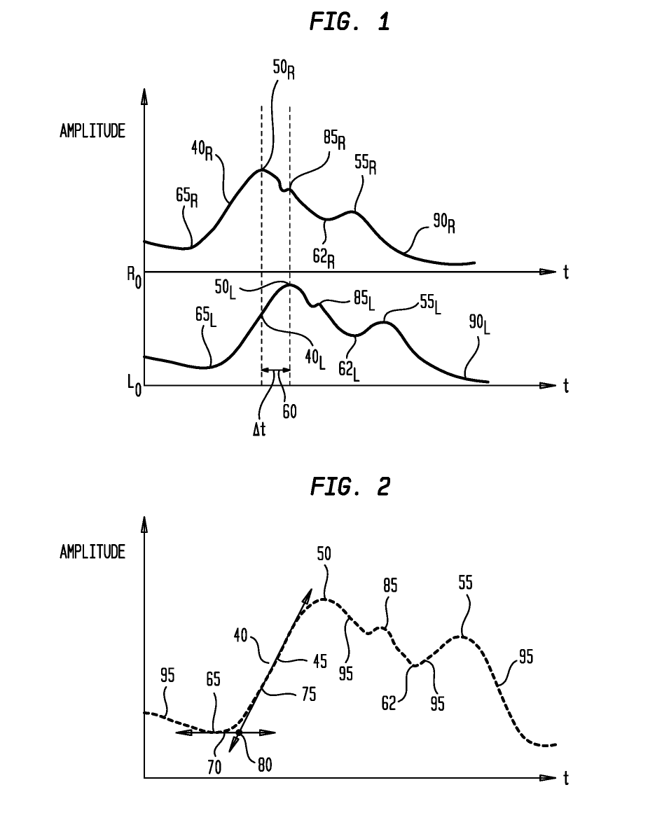

In theory, the pressure wave generated by contraction of a heart will arrive at different times at distal locations because of the variable distances traversed by the pressure wave (or pulse). Blood exiting the heart first enters the ascending aorta and then follows a number of arterial paths, beginning with the brachiocephalic (innominate) (which will further branch to form the right radial artery and right carotid artery), followed by the left common carotid artery and the left subclavian artery (which further branches to form the left radial artery), followed by the descending aorta. This arterial anatomy leads to the arterial pulse wave arriving at locations along the right arteries before arriving at corresponding (or symmetric) locations along the left arteries, i.e., the left pulse is delayed, thereby giving rise to differential pulse arrival times at symmetrical right and left locations along the head, neck, and upper extremities, e.g., the pressure wave arrives at the right radial artery before the left radial artery. Such a representative differential pulse arrival time is illustrated in FIG. 1.

FIG. 1 is a graphical diagram illustrating a representative, respective amplitudes over time of representative right (90_{R}) and left (90_{L}) arterial pressure waves, and a corresponding DPAT (60), such as from representative photoplethysmographs (“PPGs”), which may be obtained at symmetrical right (R) and left (L) locations in the neck, ears or upper extremities of an individual. The representative DPAT is illustrated in FIG. 1 by the time difference in arrival between the respective systolic peaks (50_{R} and 50_{L}) of the right and left arterial pressure waves, illustrated as DPAT time interval (Δt) 60. FIG. 1 also illustrates several other features of a representative arterial pressure wave. Each right and left arterial pressure wave generally includes a systolic peak (50_{R} and 50_{L}), a rising edge (40_{R} and 40_{L}) of the systolic peak 50, a diastolic peak (55_{R} and 55_{L}), one or more aortic-abdominal or other reflections (85_{R} and 85_{L}) typically indicating reflections of the pressure wave, a dicrotic notch (62_{R} and 62_{L}) indicating the end of systole, and a diastolic minimum (65_{R} and 65_{L}) prior to the systolic peak (50_{R} and 50_{L}). As discussed in greater detail below, any such corresponding features along the right and left arterial pressure waves (90_{R} and 90_{L}) may be utilized for the DPAT measurements or determinations, in addition to the respective systolic peaks (50_{R} and 50_{L}).

Among other advantages of DPAT over PTT measurements for BP measurement or estimation include, for example and without limitation, that the DPAT measurements in accordance with the representative embodiments does not require an ECG measurement, and further eliminates the unknown electromechanical temporal separation between contraction and generation of the pulse wave as previously mentioned. Further, the DPAT measurements in accordance with the representative embodiments also eliminates the need to grossly estimate distance between pulse generation at the heart and the distal location by recording the pulse arrival at symmetrical locations independent of distance travelled. Finally, as discussed in greater detail below, DPAT measurements in accordance with the representative embodiments can be recursively calibrated for each individual, both at rest and under various other conditions, including calibration for hydrostatic and hydrodynamic conditions which may affect DPAT measurements, and including calibration of DPAT measurements for other events which influence blood pressure.

FIG. 2 is a graphical diagram illustrating a plurality of digital samples 95 of amplitudes (over time) of a representative pressure wave 90 obtained at a location in the neck, ear, or upper extremity of an individual, illustrated as a dotted line with each dot being a corresponding digital sample, and further illustrates several features of an arterial pressure wave, including a BP waveform “foot” feature 80 (of the diastolic minimum 65) which also may be utilized for DPAT measurements or determinations (and may generally be more accurate for DPAT measurements or determinations compared to use of other features of an arterial pressure wave). As illustrated in FIG. 2, a line 70 may be defined by the diastolic minimum 65, as a tangent line having a slope equal to zero (i.e., the tangent line to the curve representing the pressure wave 95 at the diastolic minimum 65), namely, where the first derivative with respect to time at the diastolic minimum 65 is about equal to zero. Also as illustrated in FIG. 2, a line 75 may be defined by the maximum rate of increasing change in the pressure wave at the rising edge of the systolic peak 50, as a tangent line (i.e., the tangent line 75 to the curve representing the pressure wave 95 along the rising edge of the systolic peak 50) where the first derivative with respect to time of the rising edge of the systolic peak 50 is at about a maximum, illustrated at point 45 of the curve representing the rising edge of the systolic peak 50 of the pressure wave 95. The BP waveform foot feature of the pressure wave may be defined as the point of intersection of these two tangent lines 70 and 75, illustrated in FIG. 2 as BP waveform foot feature 80 (or point 80). In addition to the intersecting tangent method described above, other known methods of determining the location of the diastolic minimum 65 or the BP waveform foot feature 80 of the diastolic minimum 65 may be utilized equivalently, including for example and without limitation: the maximum first derivative with respect to time between the diastolic minimum 65 and the systolic peak 50; the maximum second derivative with respect to time between the diastolic minimum 65 and the maximum first derivative with respect to time; and a fraction of the pulse pressure.

In a representative embodiment, corresponding BP waveform foot features (80) of the right and left pressure waves, from measurements obtained at symmetrical right and left positions (or locations) on or at the neck, ear, or upper extremity of an individual, are utilized for DPAT measurements or determinations, particularly at elevated BP conditions, as it is less subject to noise and the impact of other wave reflections. In another representative embodiment, corresponding systolic peaks (50R and 50L) of the right and left pressure waves, also from measurements obtained at symmetrical right and left positions (or locations) on or at the neck, ear, or upper extremity of an individual, are utilized for DPAT measurements or determinations. In yet another representative embodiment, corresponding points (45) of the maximum rate of increasing change in the right and left pressure waves, also from measurements obtained at symmetrical right and left positions (or locations) on or at the neck, ear, or upper extremity of an individual, are utilized for DPAT measurements or determinations. In yet another representative embodiment, a predetermined percentage (e.g., 50% or 75%, for example and without limitation) of the rising edge 40 (pressure increase) leading to the respective systolic peaks (50R and 50L) in the right and left pressure waves, also from measurements obtained at symmetrical right and left positions (or locations) on or at the neck, ear, or upper extremity of an individual, are utilized for DPAT measurements or determinations.

In yet another representative embodiment, ratios of amplitudes of various features of the right and left pressure waves, also from measurements obtained at symmetrical right and left positions (or locations) on or at the neck, ear, or upper extremity of an individual, are utilized for BP measurements or estimations. For example and without limitation, a ratio of the amplitude of the systolic peak 50_{R} to the amplitude of the aortic-abdominal reflection 85_{R}, for right pressure wave 90_{R}, may be compared to a ratio of the amplitude of the systolic peak 50_{L} to the amplitude of the aortic-abdominal reflection 85_{L}, for left pressure wave 90_{L}, may be utilized as an indicator of BP.

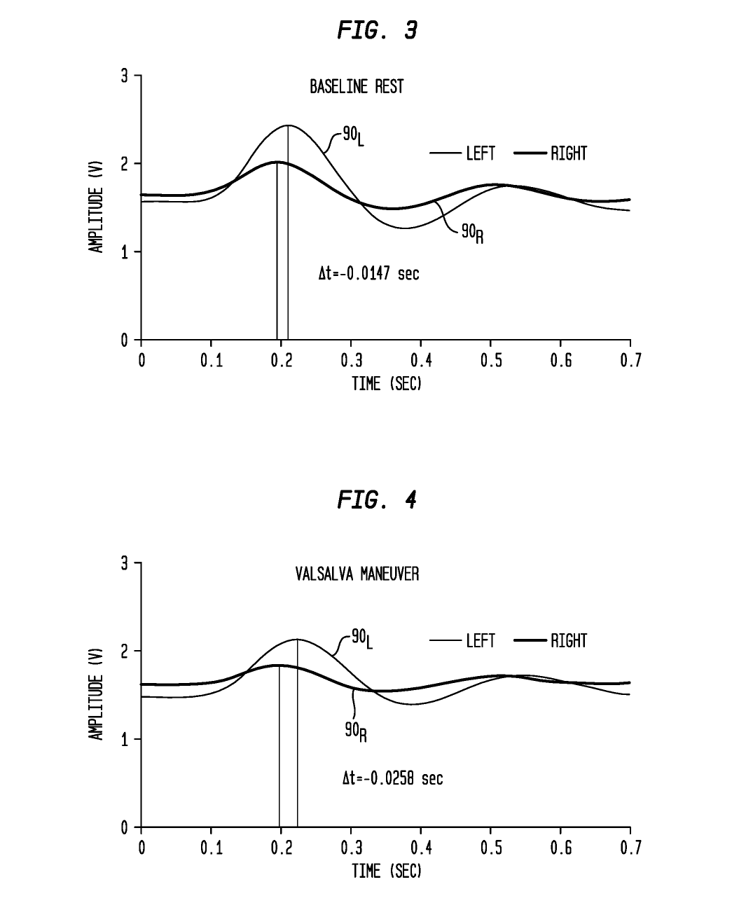

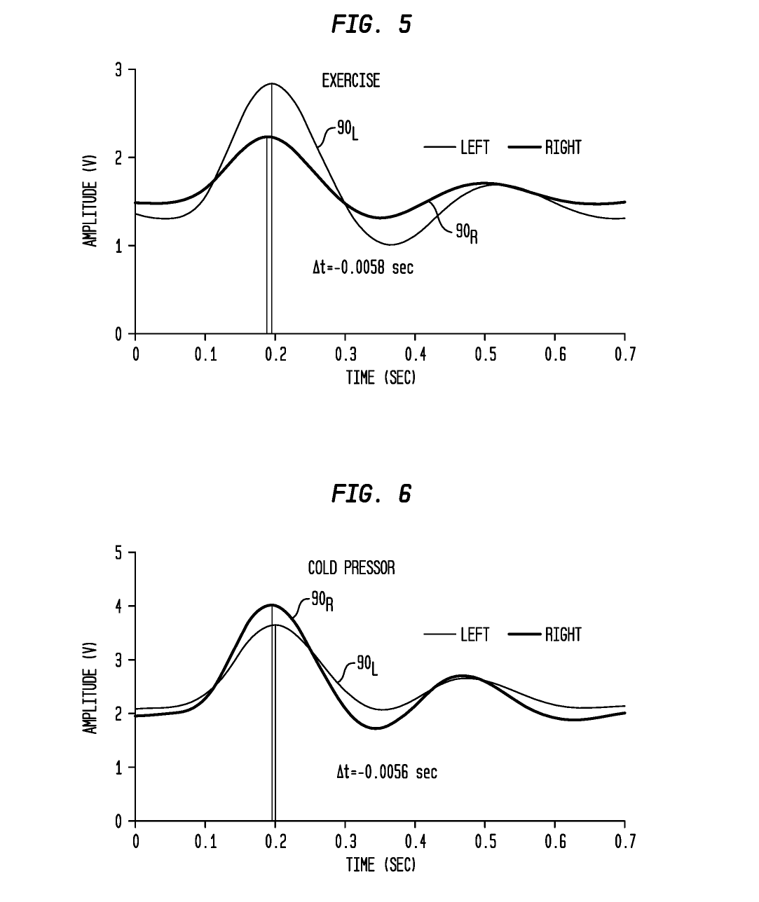

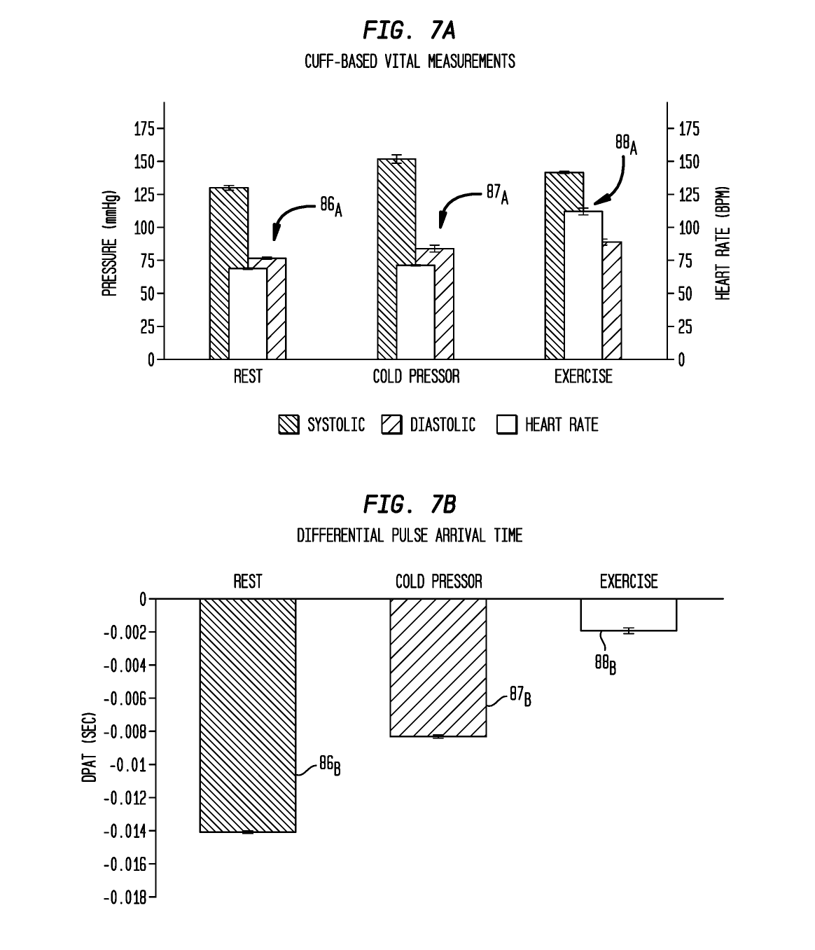

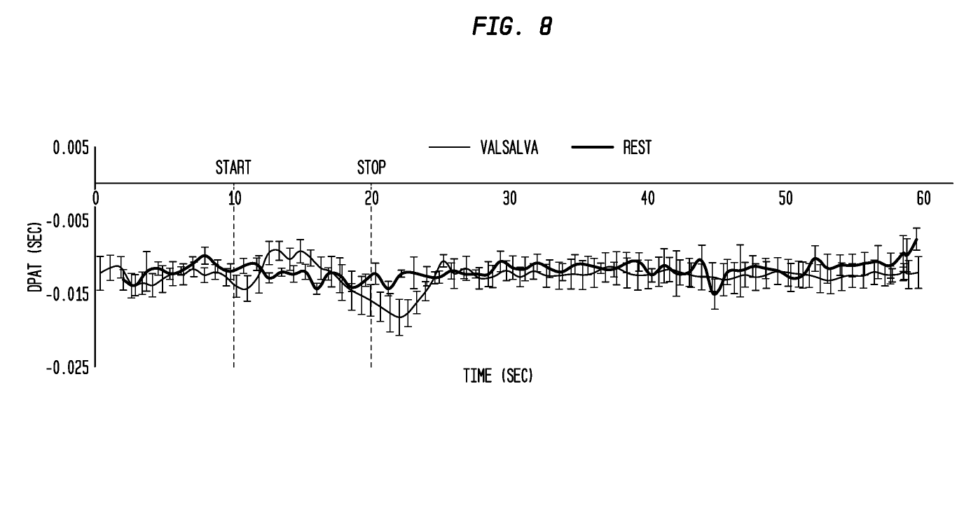

The DPAT is inversely proportional to the systemic blood pressure, with a higher blood pressure resulting in a symmetrically (right and left) increased arterial pulse velocity, which reduces the difference between the right and left pulse arrival times. This inverse relationship is illustrated in FIGS. 3-7. FIG. 3 is a graphical diagram illustrating a baseline differential pulse arrival time from representative right and left arterial pressure waves (90_{R} and 90_{L}) obtained at symmetrical right and left locations or positions in the neck, ears or upper extremities of an individual when the individual is at rest. FIG. 4 is a graphical diagram illustrating an increased differential pulse arrival time from representative right and left arterial pressure waves (90_{R} and 90_{L}) obtained at symmetrical right and left locations or positions in the neck, ears or upper extremities of an individual, following performance of a Valsalva maneuver, which lowers BP. FIG. 5 is a graphical diagram illustrating a decreased differential pulse arrival time from representative right and left arterial pressure waves obtained at symmetrical right and left locations or positions in the neck, ears or upper extremities of an individual, following exercise, which increases blood pressure. FIG. 6 is a graphical diagram illustrating a decreased differential pulse arrival time from representative right and left arterial pressure waves (90_{R} and 90_{L}) obtained at symmetrical right and left locations or positions in the neck, ears or upper extremities of an individual, following a cold pressor test, which also increases blood pressure. FIG. 7A is a bar chart diagram illustrating baseline blood pressures of individuals at rest (86_{A}), and increased blood pressures of individuals following a cold pressor test (87_{A}) and following exercise (88_{A}). FIG. 7B is a bar chart diagram illustrating a baseline DPAT of an individual at rest (86_{B}), and corresponding decreased differential pulse arrival times from representative right and left arterial pressure waves obtained at symmetrical right and left locations or positions in the neck, ears or upper extremities of the individual following a cold pressor test (87_{B}) and following exercise (88_{B}).

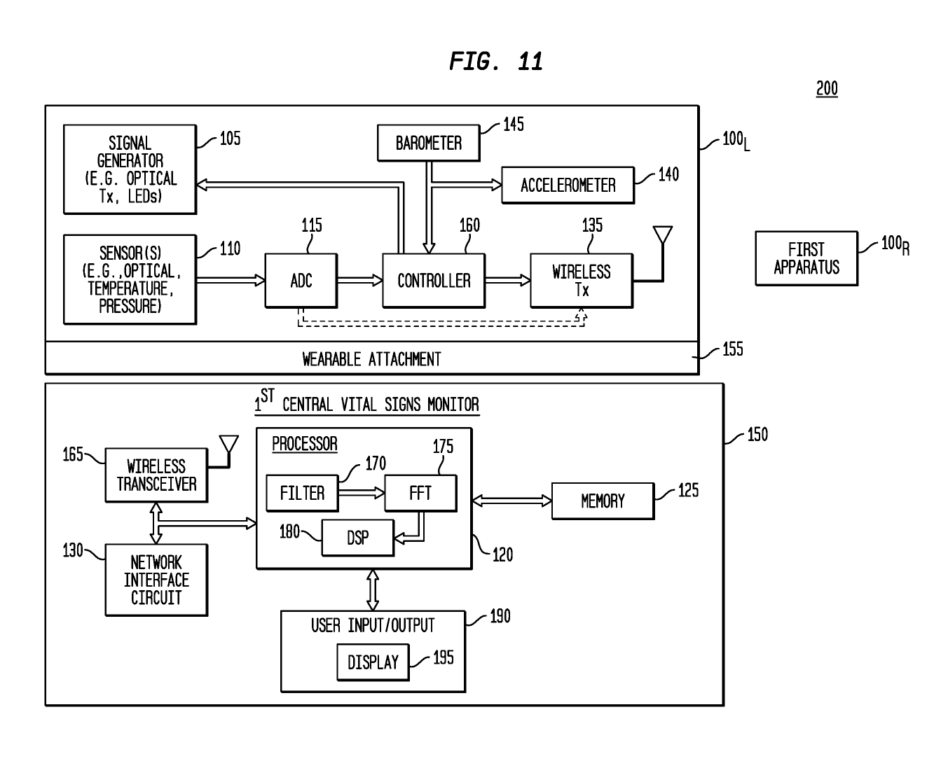

FIG. 11 is a block diagram of representative first apparatus 100 and first system 200 embodiments. As illustrated in FIG. 11, two generally identical first apparatuses 100 are utilized in the first system 200, illustrated as first apparatus 100_{L} and first apparatus 100_{R}, which are respectively utilized to acquire measurements or data, from symmetrical left and right locations or positions in the neck, ears or upper extremities of the individual, utilized in DPAT measurements or determinations. The first apparatus 100_{L} and first apparatus 100_{R} differ only insofar as one receives measurements or data from the individual's left side and the other receives measurements or data from the individual's right side, and are otherwise are identical, interchangeable, and function identically; as a result, without a loss of generality or specificity, the first apparatus 100_{L} and first apparatus 100_{R} are individually and collectively equivalently referred to as a first apparatus 100. The first system 200 further comprises a first central vital signs monitor 150, which receives the measurements or data from each of the first apparatus 100_{L} and first apparatus 100_{R}, generates DPAT measurements or determinations, and provides corresponding estimates of measurements of blood pressure and other vital signs, as mentioned above.

It should be noted that the first central vital signs monitor 150 (and the second central vital signs monitor 250 discussed below) are “central” in the sense of being the main, predominant or principal receivers of the signals from the apparatus 100, 500 and the providers of corresponding estimates of measurements of blood pressure and other vital signs, and not “central” in terms of determining a “central blood pressure”.

Each of the first apparatus 100_{L} and first apparatus 100_{R} comprises a signal generator 105, one or more sensor(s) 110, an analog-to-digital converter (ADC) 115, and a wireless transmitter 135. The signal generator 105, such as an optical transmitter (e.g., a plurality of light emitting diodes), generates a signal (such as electrical, light, acoustic or pressure) for transmission to locations or positions in the neck, ears or upper extremities of the individual, such as light emission in a first selected wavelength band. The one or more sensor(s) 110 (such as optical sensor(s), acoustic sensor(s) (e.g., one or more microphones), surface acoustic sensor(s), pressure sensor(s)), bioimpedance sensor(s), temperature sensor(s), and so on, receives a return or sensed signal which is indicative of an arterial pressure wave (90_{R} or 90_{L}), such as light in a second selected wavelength band or sound, generally reflected from locations or positions in the neck, ears or upper extremities of the individual, and generate a corresponding analog sensor electrical signal. The analog-to-digital converter (ADC) 115 samples the analog sensor electrical signal from the one or more sensor(s) 110 and generates a stream or series of corresponding digital amplitude values, each of which is indicative or represents the amplitude of the arterial pressure waves (90_{R} and 90_{L}) during the sampling time interval, such as the sampled digital values illustrated and discussed above with reference to FIG. 2. The wireless transmitter 135 wirelessly transmits the corresponding stream or series of corresponding digital amplitude values to the first central vital signs monitor 150.

Optionally, each of the first apparatus 100_{L} and first apparatus 100_{R} may also include an accelerometer 140, a barometer 145, a controller 160, and a wearable attachment 155. When included, the wearable attachment 155 may be a wristband, a ring for a finger, a finger sleeve, a glove, an ear clip, or a reposable or reusable adhesive material, for example and without limitation. When included, the accelerometer 140 measures or determines movement of the individual, and generates and provides to the controller 160 corresponding movement data. Also when included, a barometer 145 measures or determines elevation (or elevation changes) of the individual, such as raising or lowering an arm, and generates and provides to the controller 160 corresponding elevation data. Such movement and/or elevation data may be utilized by the first central vital signs monitor 150 to generate corresponding estimates of measurements of BP reflecting such movement or changes in elevation, such as changes in the position of the individual which affect DPAT measurements or determinations and may be accounted for in the corresponding estimates of measurements of blood pressure. For this first system 200, the controller combines the stream or series of corresponding digital values (indicative of the arterial pressure waves (90_{R} or 90_{L}), with the movement data and/or elevation data, for wireless transmission by the wireless transmitter 135 to the first central vital signs monitor 150.

As discussed in greater detail below, in representative embodiments in which a wearable attachment 155 is included, each of the first apparatus 100_{L} and first apparatus 100_{R} are placed into symmetrical locations or positions in the neck, ears or upper extremities and may be worn by the individual. In other representative embodiments which do not include a wearable attachment 155, also for example and without limitation, both the first apparatus 100_{L} and first apparatus 100_{R} may be arranged together in a housing, as illustrated and discussed below, such as a handheld device, a case for a smartphone, and so on. For such an arrangement, the individual holds the housing to contact a respective fingertip of the right hand and fingertip of the left hand with the corresponding one or more right and left sensor(s) 110, to generate the data for the DPAT measurements or determinations, such as whenever an individual is holding the smartphone to check their email or messages, for example and without limitation.

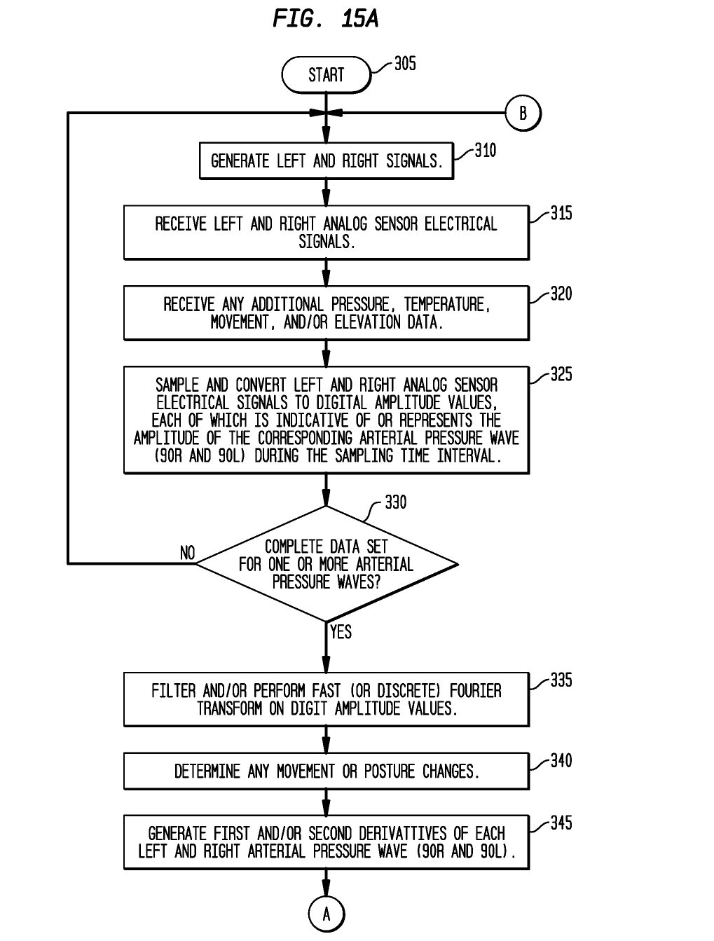

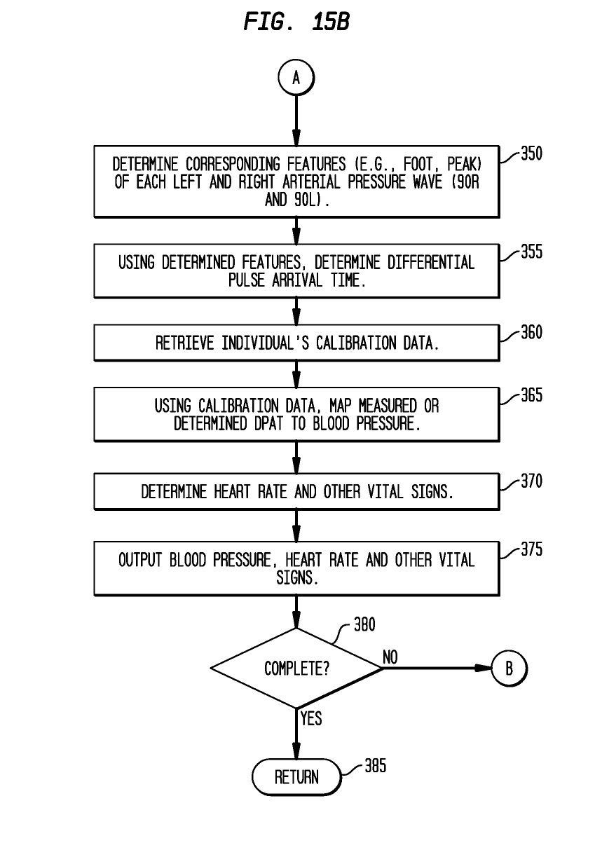

The first central vital signs monitor 150 generally comprises a wireless transceiver (or receiver and transmitter) 165, a processor 120, a memory 125, a network interface circuit 130, and a user input and output device 190, such as a touch screen display 195 or any other type of visual display, for example. The memory 125 generally stores calibration data, as discussed in greater detail below, and may also store collected data and corresponding results, such as DPAT measurements or determinations and corresponding estimates or measurements of the BP and other vital signs of the individual. The wireless transceiver 165, which may be included in the network interface circuit 130, receives the stream or series of corresponding digital amplitude values indicative of or representing the arterial pressure waves (90_{R} or 90_{L}), and possibly also any movement data and/or elevation data, from each of the first apparatus 100_{L} and first apparatus 100_{R}, and provides or transfers this data to the processor 120. Using this stream or series of corresponding digital amplitude values (indicative of or representing the arterial pressure waves (90_{R} or 90_{L}), along with any movement data and/or elevation data, the processor 120 generates the DPAT measurements or determinations and corresponding estimates or measurements of the BP and other vital signs of the individual. As discussed in greater detail below with reference to the flow chart of FIG. 15, the processor 120 may also be considered to include, such as through configuration or programming, a filter 170, a fast Fourier transform (or discrete Fourier transform) circuit or block 175, and a digital signal processor (“DSP”) or DSP block 180.

The processor 120 may then provide the estimates or measurements of the BP and other vital signs of the individual to the user input and output device 190, such as for display to the individual on a touch screen display 195. The processor 120 also may then provide the estimates or measurements of the BP and other vital signs of the individual to the network interface circuit 130, such as for transmission of the estimates or measurements of the BP and other vital signs of the individual to another location or device, such as to a hospital or clinic computing system, also for example and without limitation.

Not separately illustrated in FIG. 11, those having skill in the art will recognize that devices such as first central vital signs monitor 150, first apparatus 100_{L} and first apparatus 100_{R} also generally include clocking circuitry and distribution, and a power supply with power distribution, which may be a battery or other energy source, for example and without limitation.

Those having skill in the art also will recognize that for whatever type of signal generator 105 is selected for a given embodiment, such as electrical, optical, sound, pressure, etc., a corresponding type of sensor(s) 110 for signal acquisition is or are also then selected, such as optical sensor(s) 110, one or more microphones as acoustic sensor(s) 110, a pressure sensor(s) 110, bioimpedance sensor(s) detecting electrical signals, temperature sensor(s), for example and without limitation. It should also be noted that depending upon the type of sensing selected, a signal generator 105 may become optional and is not required, such as for bioimpedance sensing and temperature sensing, also for example and without limitation. All of these variations are considered equivalent and within the scope of the disclosure, and further apply to the other apparatus 300, 500, 700 and system 400, 600 (and/or 700) embodiments discussed below.

Optical signal generators 105 and optical sensor(s) 110 may be utilized in a selected embodiment of a first apparatus 100, to generate photoplethysmography (“PPG”) data which will be utilized for DPAT measurements or determinations and corresponding estimates or measurements of the BP and other vital signs of the individual. For example and without limitation, one or more optical signal generators 105 may comprise a plurality of light emitting diodes (“LEDs”), such as LEDs which emit light in a first wavelength band including about 520 nm. As an arterial pulse propagates, blood volume increases and additional red blood cells are present which increase the absorption of green wavelengths, decreasing the amount of light reflected back from the locations or positions in the neck, ears or upper extremities of the individual, providing an indication or representation of the arterial pressure waves (90_{R} or 90_{L}). Optical sensor(s) 110 are then utilized to detect the reflected light, typically in a band of about 520 nm-560 nm, for example and without limitation. The other apparatus 300, 500, 700 and system 400, 600 (and/or 700) embodiments discussed below may also include generation of PPG data.

In a representative embodiment of a first apparatus 100, multiple types of sensor(s) 110 are utilized (and further apply to the other apparatus 300, 500, 700 and system 400, 600 (and/or 700) embodiments discussed below). In addition to an optical sensor 110 for obtaining PPG data, a temperature sensor 110 and a pressure sensor 110 are also utilized, to provide greater accuracy in converting, transforming or otherwise mapping DPAT measurements or determinations to absolute measurements of the BP and other vital signs of the individual. When arterial vessels may be constricted or dilated, such as when an individual's hands are cold or warm, respectively, arterial pressure waves (90_{R} or 90_{L}) and corresponding DPAT measurements or determinations may be affected without corresponding actual changes in the subject's absolute BP. Similarly, the contact pressure exerted by the first apparatus 100 on the subject individual may also affect the amplitude of the arterial pressure waves (90_{R} or 90_{L}) and resulting DPAT measurements or determinations, again without corresponding changes in the subject's absolute BP, such as when a wearable attachment 155 is included or the subject individual applies pressure to the first apparatus 100 during use. Accordingly, during a calibration process as discussed in greater detail below, temperature and pressure data, along with DPAT measurements or determinations, are included in the overall calibration of an individual's DPAT (measured or determined with representative systems 200, 400, 600, and 700) with his or her BP (independently measured, such as using a cuff-based system), under various conditions and events. This calibration data will generally include DPAT measurements or determinations, along with temperature and pressure data, and typically cuff-based measurements of the subject's absolute BP. The calibration data (stored in a memory 125) are then utilized during operation of a system 200, 400, 600, 700 in which the subject's temperature, contact pressure, and DPAT are measured or otherwise determined, and then converted, transformed or mapped to the subject's BP, to provide a more accurate estimate or measurement of the BP and other vital signs of the subject individual.

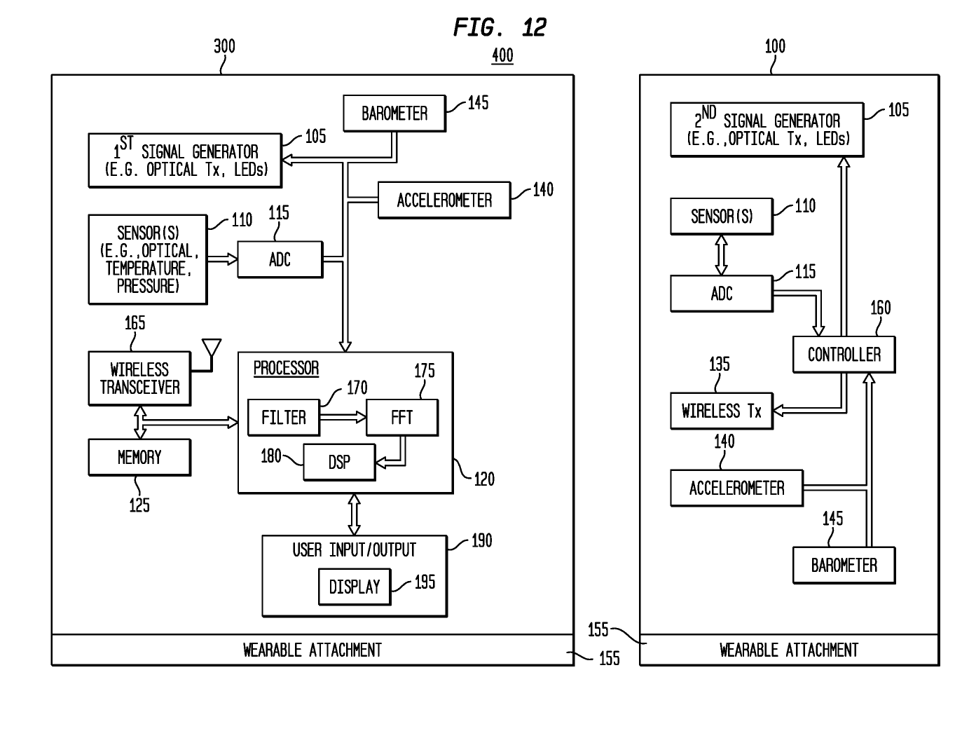

FIG. 12 is a block diagram of representative second apparatus 300 and second system 400 embodiments. As illustrated in FIG. 12, a second system 400 generally comprises a second apparatus 300 in conjunction with a first apparatus 100, both of which are respectively utilized to acquire measurements or data, from symmetrical left and right locations or positions in the neck, ears or upper extremities of the individual, utilized in DPAT measurements or determinations. For example and without limitation, in a second system 400, a second apparatus 300 may be worn on a left wrist and the first apparatus 100 may be worn on a right wrist, or vice-versa. The first apparatus 100 operates as described above with reference to FIG. 11. The second apparatus 300 operates as described above for the first apparatus 100 and further comprises many of the components and functionality of a first central vital signs monitor 150. Accordingly, the second apparatus 300 also generates measurements or data from a selected left or right location or position in the neck, ears or upper extremities of the individual, but also receives the measurements or data from the first apparatus 100 from, respectively, a symmetrical right or left location or position in the neck, ears or upper extremities of the individual, and further generates DPAT measurements or determinations and provides corresponding estimates of measurements of blood pressure and other vital signs, as discussed above.

The second system 400 may be viewed as combining the components and functionality of many (but generally not all) the components and functions of the first system 200 into two devices (a second apparatus 300 and a first apparatus 100), rather than distributing these components and functions between and among three devices (first apparatus 100_{L}, first apparatus 100_{R}, and first central vital signs monitor 150). The second system 400 also eliminates components that could now be considered redundant, optional or unnecessary when selected components and functions of the first central vital signs monitor 150 are included in the second apparatus 300 (e.g., eliminating a controller 160 and wireless transmitter 135 in the second apparatus 300, and optionally eliminating a network interface circuit 130 in the second apparatus 300). Accordingly, unless specified to the contrary, the components of the second system 400 generally function identically to the components of the first system 200 described above.

Accordingly, the components of the second system 400 embodiment are asymmetric, using a first apparatus 100 and a second apparatus 300, with the second apparatus 300 generally including or combining the overall functionality of a first apparatus 100 and a first central vital signs monitor 150, without redundancy.

The second apparatus 300 also comprises a signal generator 105, one or more sensor(s) 110, and an analog-to-digital converter (ADC) 115, all of which function as discussed above. Optionally, the second apparatus 300 may also include an accelerometer 140, a barometer 145, and a wearable attachment 155, all of which function as discussed above.

The second apparatus 300 also generally comprises a wireless transceiver (or receiver and transmitter) 165, a processor 120, a memory 125, and a user input and output device 190, such as a touch screen display 195 or any other type of visual display, an on/off button, and so on, also for example, all of which function as discussed above. Optionally, the second apparatus 300 may include a network interface circuit 130. The memory 125 of the second apparatus 300 also generally stores calibration data, as discussed in greater detail below, and may also store collected data and corresponding results, such as DPAT measurements or determinations and corresponding estimates or measurements of the BP and other vital signs of the individual. The wireless transceiver 165 of the second apparatus 300 receives the stream or series of corresponding digital amplitude values indicative of or representing the arterial pressure waves (90_{R} or 90_{L}), and possibly also any movement data and/or elevation data, from the first apparatus 100 in the second system 400, and provides or transfers this data to the processor 120 of the second apparatus 300. The digital amplitude values indicative of or representing the arterial pressure waves (90_{L} or 90_{R}) generated by the analog-to-digital converter (ADC) 115, from the corresponding analog sensor electrical signal provided by sensor(s) 110 of the second apparatus 300, are also transferred to the processor 120 of the second apparatus 300. Using this stream or series of corresponding digital amplitude values (indicative of or representing the arterial pressure waves (90_{R} or 90_{L}), along with any movement data and/or elevation data, from symmetrical locations or positions in the neck, ears or upper extremities of the individual, the processor 120 of the second apparatus 300 also generates the DPAT measurements or determinations and corresponding estimates or measurements of the BP and other vital signs of the individual, as discussed above. Also as discussed in greater detail below with reference to the flow chart of FIG. 15, the processor 120 may also be considered to include, such as through configuration or programming, a filter 170, a fast Fourier transform (or discrete Fourier transform) circuit or block 175, and a digital signal processor (“DSP”) or DSP block 180.

The processor 120 may then provide the estimates or measurements of the BP and other vital signs of the individual to the user input and output device 190 of the second apparatus 300, such as for display to the individual on a touch screen or other display 195. For example, in a representative embodiment in which the the second apparatus 300 is worn on a left or right wrist by a subject individual, using a wristband or bracelet as a wearable attachment 155, the individual's BP and other vital signs may be displayed and viewed by the user in real time similarly or equivalently to reading a wristwatch. Also not separately illustrated in FIG. 12, those having skill in the art will recognize that devices such as the first apparatus 100 and second apparatus 300 also generally include clocking circuitry and distribution, and a power supply with power distribution, which may be a battery or other energy source, for example and without limitation.

It should be noted that any of the systems 200, 400, 600, 700 may be utilized in conjunction with other devices and systems, as known in the computer and communications fields, such as optional relay stations or docking units, not separately illustrated. For example and without limitation, such an optional relay station or docking unit may receive DPAT or BP measurements or determinations from a second apparatus 300, and transfer this data to a network or cloud storage device (also not separately illustrated), which also may be accessed by physicians or other clinical staff, such as through a compatible portal at a hospital or a clinical computing system.

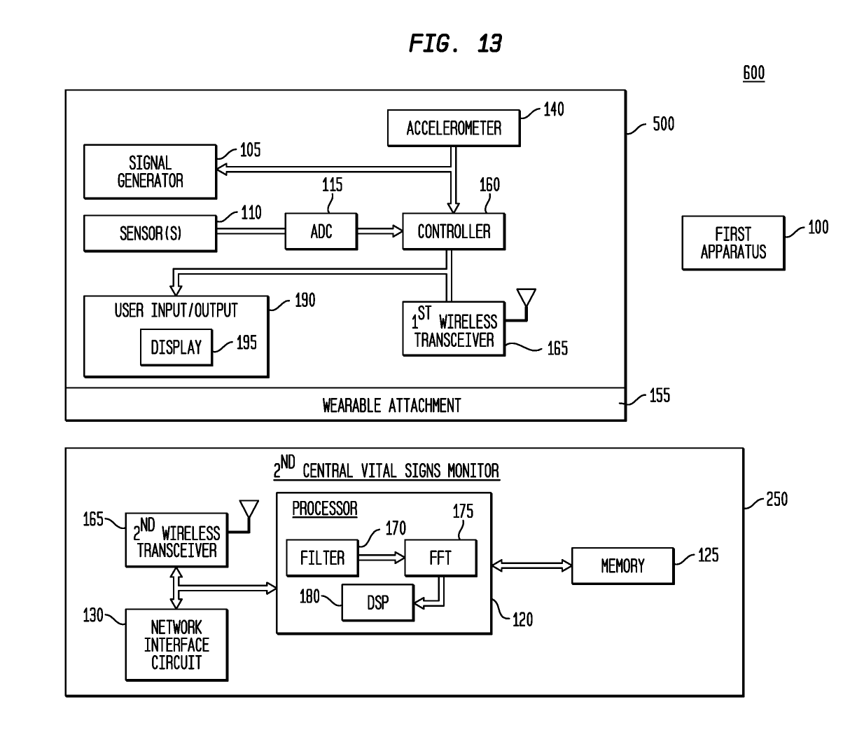

FIG. 13 is a block diagram of representative third apparatus 500 and third system 600 embodiments. As illustrated in FIG. 13, a third system 600 generally comprises a third apparatus 500 in conjunction with a first apparatus 100 and a second central vital signs monitor 250. The third apparatus 500 and first apparatus 100 are respectively utilized to acquire measurements or data, from symmetrical left and right locations or positions in the neck, ears or upper extremities of the individual, utilized in DPAT measurements or determinations. For example and without limitation, in a third system 600, a third apparatus 500 may be worn on a left wrist and the first apparatus 100 may be worn on a right wrist, or vice-versa. The first apparatus 100 operates as described above with reference to FIG. 11. The third apparatus 500 operates as described above for the first apparatus 100 and further comprises two additional components and functions of a first central vital signs monitor 150, namely, the third apparatus 500 further comprises a first wireless transceiver (or receiver and transmitter) 165 (in lieu of a wireless transmitter 135), and a user input and output device 190, such as a touch screen display 195 or any other type of visual display, an on/off button, and so on, also for example, all of which function as discussed above. Accordingly, the third apparatus 500 also generates measurements or data from a selected left or right location or position in the neck, ears or upper extremities of the individual, and transmits the digital amplitude values, indicative of or representing the arterial pressure waves (90_{L} or 90_{R}) generated by the analog-to-digital converter (ADC) 115, from the corresponding analog sensor electrical signal provided by sensor(s) 110 of the third apparatus 500, to the second central vital signs monitor 250, which in turn generates DPAT measurements or determinations and provides corresponding estimates of measurements of blood pressure and other vital signs, as discussed above.

The third system 600 may be viewed as combining the components and functionality of many (but generally not all) the components and functions of the first system 200, as a different combination or distribution into three devices, a first apparatus 100, a third apparatus 500, and a second central vital signs monitor 250. Accordingly, unless specified to the contrary, the components of the third system 600 generally function identically to the components of the first system 200 described above.

The third apparatus 500 also comprises a signal generator 105, one or more sensor(s) 110, and an analog-to-digital converter (ADC) 115, all of which function as discussed above. Optionally, the third apparatus 500 may also include an accelerometer 140, a barometer 145 (not separately illustrated), and a wearable attachment 155, all of which function as discussed above.

The third apparatus 500 also generally comprises a wireless transceiver (or receiver and transmitter) 165, a controller 160, and a user input and output device 190, such as a touch screen display 195 or any other type of visual display, an on/off button, and so on, also for example, all of which function as discussed above. For this third apparatus 500 embodiment, the controller 160 also operates as a display controller to provide first control signals to the user input and output device 190, to display the corresponding estimates of measurements of blood pressure and other vital signs, further provides second control signals to the first wireless transceiver (or receiver and transmitter) 165, and may also provide control signals to the signal generator 105 of the third apparatus 500. The first wireless transceiver 165 of the third apparatus 500 transmits the stream or series of corresponding digital amplitude values indicative of or representing the arterial pressure waves (90_{R} or 90_{L}) (as generated by the sensor(s) 110 and an analog-to-digital converter (ADC) 115 of the third apparatus 500), and possibly also any movement data and/or elevation data, to the second central vital signs monitor 250.

Using this stream or series of corresponding digital amplitude values (indicative of or representing the arterial pressure waves (90_{R} or 90_{L}), along with any movement data and/or elevation data, from symmetrical locations or positions in the neck, ears or upper extremities of the individual, from both the first apparatus 100 and the third apparatus 500, the processor 120 of the second central vital signs monitor 250 also generates the DPAT measurements or determinations and corresponding estimates or measurements of the BP and other vital signs of the individual, as discussed above. Also as discussed in greater detail below with reference to the flow chart of FIG. 15, the processor 120 may also be considered to include, such as through configuration or programming, a filter 170, a fast Fourier transform (or discrete Fourier transform) circuit or block 175, and a digital signal processor (“DSP”) or DSP block 180.

The processor 120 of the second central vital signs monitor 250 may then provide the estimates or measurements of the BP and other vital signs of the individual to the second wireless transceiver 165 for transmission to the third apparatus 500 (via first wireless transceiver 165) for display to the user via the user input and output device 190 of the third apparatus 500, such as for display to the individual on a touch screen or other display 195. For example, in a representative embodiment in which the second apparatus 300 is worn on a left or right wrist by a subject individual, using a wristband or bracelet as a wearable attachment 155, the individual's BP and other vital signs may be displayed and viewed by the user in real time similarly or equivalently to reading a wristwatch. Also not separately illustrated in FIG. 13, those having skill in the art will recognize that devices such as the first apparatus 100, third apparatus 500, and second central vital signs monitor 250 also generally include clocking circuitry and distribution, and a power supply with power distribution, which may be a battery or other energy source, for example and without limitation.

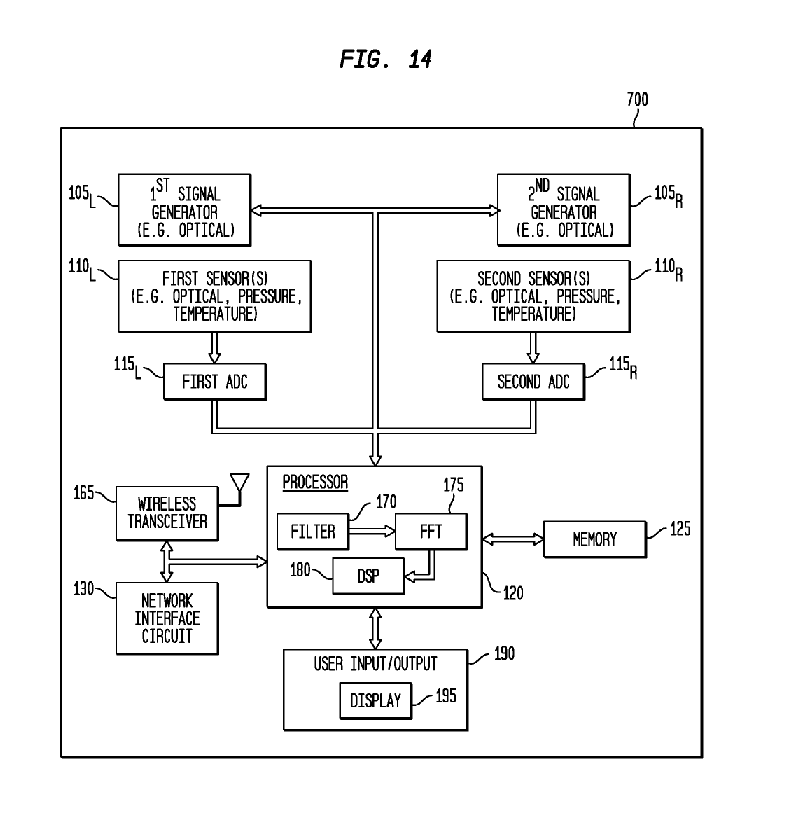

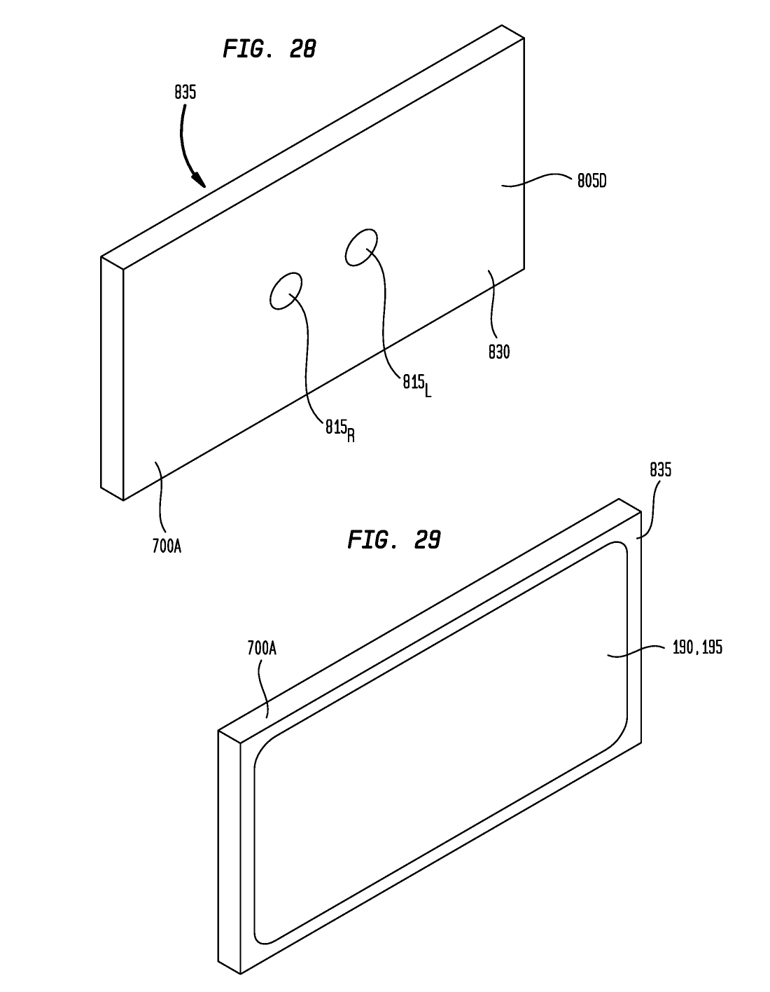

FIG. 14 is a block diagram of a representative fourth combined apparatus and system 700 embodiment, which may be referred to equivalently as a fourth apparatus 700 and/or a fourth system 700, as most (but not all) of the components and functionality described above are included in a single device (typically inside a housing, not separately illustrated in FIG. 14, but illustrated below with reference to FIGS. 28 and 29). The fourth apparatus 700 and/or fourth system 700 combines many of the components and functionality of two (left and right) first apparatuses 100 together with many of the components and functionality of a first central vital signs monitor 150 (and eliminates unnecessary or redundant components, as described above), as illustrated, into a single device. Accordingly, unless specified to the contrary, the components of the fourth apparatus 700 and/or fourth system 700 generally function identically to the components of the first, second and third systems 200, 400, 600 described above.

This representative fourth apparatus 700 and/or fourth system 700 is designed to be a singular, hand-held device, which may either have its own housing or may be integrated into a housing utilized with another, second device or article of manufacture, such as a smartphone or tablet computer case or housing. For operation of this representative fourth apparatus 700 and/or fourth system 700, a subject individual will hold the fourth apparatus 700 and/or fourth system 700 in both hands, typically at about heart level, and generally place (symmetrically) left and right fingers into corresponding positions or locations in the housing (as illustrated and discussed below). This is highly advantageous in reducing noise levels and potential sources of error from motion and hydrostatic or hydrodynamic effects. As a result, an accelerometer 140 and/or a barometer 145 are optional and generally not included in a representative fourth apparatus 700 and/or fourth system 700.

The fourth apparatus 700 is utilized to acquire measurements or data, from symmetrical left and right locations or positions in the upper extremities of the individual, typically hands or fingers, utilized in DPAT measurements or determinations. The fourth apparatus 700 and/or fourth system 700 comprises first and second signal generators 105_{L} and 105_{R}, first and second sensor(s) 110_{L} and 110_{R}, first and second analog-to-digital converters (ADC) 115 _{L} and 115_{R}, a wireless transceiver (or receiver and transmitter) 165, a processor 120, a memory 125, a network interface circuit 130, and a user input and output device 190, such as a touch screen display 195 or any other type of visual display, for example.

The first signal generator 105_{L}, such as an optical transmitter (e.g., a plurality of light emitting diodes), generates a signal (such as electrical, light, acoustic or pressure) for transmission to locations or positions in the left upper extremity of the individual (e.g., a left fingertip), such as light emission in a first selected wavelength band. The one or more first sensor(s) 110_{L} (such as optical sensor(s), acoustic sensor(s) (e.g., one or more microphones), surface acoustic sensor(s), pressure sensor(s)), bioimpedance sensor(s), temperature sensor(s), and so on, as discussed above, receives a return or sensed signal which is indicative of an arterial pressure wave (90_{L}), such as light in a second selected wavelength band or sound, generally reflected from the location or position in the left upper extremity of the individual, and generates a corresponding analog sensor electrical signal. The first analog-to-digital converter (ADC) 115_{L} also samples the analog sensor electrical signal from the first sensor(s) 110_{L} and generates a stream or series of corresponding digital amplitude values, each of which is indicative or represents the amplitude of the arterial pressure waves (90_{L}) during the sampling time interval, such as the sampled digital values illustrated and discussed above with reference to FIG. 2, which are provided to the processor 120 of the fourth apparatus 700.

Similarly, the second signal generator 105_{R}, such as an optical transmitter (e.g., a plurality of light emitting diodes), generates a signal (such as electrical, light, acoustic or pressure) for transmission to locations or positions in the right upper extremity of the individual (e.g., a right fingertip), such as light emission in a first selected wavelength band. The one or more second sensor(s) 110_{R} (such as optical sensor(s), acoustic sensor(s) (e.g., one or more microphones), surface acoustic sensor(s), pressure sensor(s)), bioimpedance sensor(s), temperature sensor(s), and so on, as discussed above, receives a return or sensed signal which is indicative of an arterial pressure wave (90_{R}), such as light in a second selected wavelength band or sound, generally reflected from the location or position in the right upper extremity of the individual, and generates a corresponding analog sensor electrical signal. The second analog-to-digital converter (ADC) 115_{R} also samples the analog sensor electrical signal from the second sensor(s) 110_{R} and generates a stream or series of corresponding digital amplitude values, each of which is indicative or represents the amplitude of the arterial pressure waves (90_{R}) during the sampling time interval, such as the sampled digital values illustrated and discussed above with reference to FIG. 2, which are provided to the processor 120 of the fourth apparatus 700.

The memory 125 of the fourth apparatus 700 also generally stores calibration data, as discussed in greater detail below, and may also store collected data and corresponding results, such as DPAT measurements or determinations and corresponding estimates or measurements of the BP and other vital signs of the individual. Using the two streams or series of corresponding digital amplitude values (indicative of or representing the arterial pressure waves (90_{R} or 90_{L}), the processor 120 generates the DPAT measurements or determinations and corresponding estimates or measurements of the BP and other vital signs of the individual. As discussed in greater detail below with reference to the flow chart of FIG. 15, the processor 120 of the fourth apparatus 700 may also be considered to include, such as through configuration or programming, a filter 170, a fast Fourier transform (or discrete Fourier transform) circuit or block 175, and a digital signal processor (“DSP”) or DSP block 180.

The processor 120 may then provide the estimates or measurements of the BP and other vital signs of the individual to the user input and output device 190, such as for display to the individual on a touch screen display 195. The processor 120 also may then provide the estimates or measurements of the BP and other vital signs of the individual to the network interface circuit 130 and/or the wireless transceiver 165 (which also may be included in the network interface circuit 130), such as for transmission of the estimates or measurements of the BP and other vital signs of the individual to another location or device, such as to a hospital or clinic computing system, also for example and without limitation.

Not separately illustrated in FIG. 14, those having skill in the art will recognize that devices such as the fourth apparatus 700 also generally include clocking circuitry and distribution, and a power supply with power distribution, which may be a battery or other energy source, for example and without limitation.

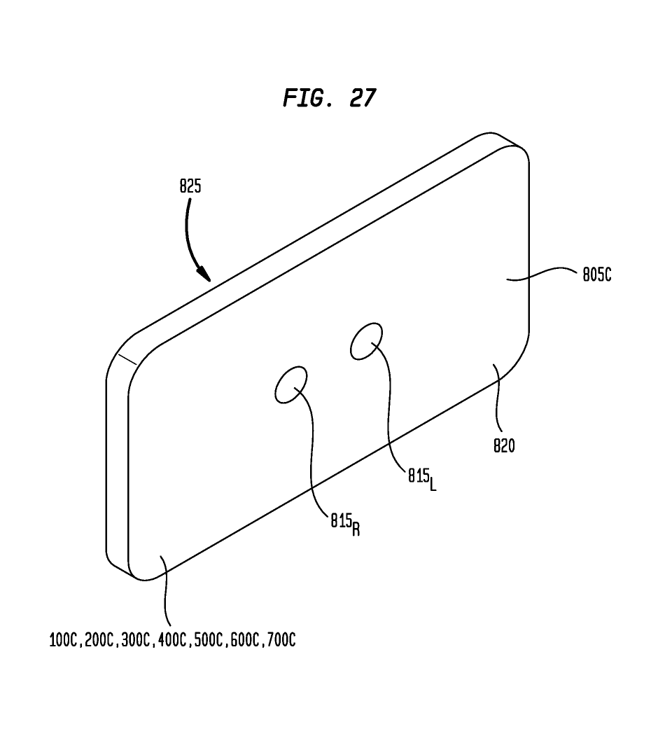

A variation of the fourth apparatus 700 is also within the scope of the present disclosure. For this variation, the first and second signal generators 105, the first and second sensors 110, and the first and second analog-to-digital converters 115 are contained in a housing (such as a housing 805C illustrated in FIG. 27), and a wireless transceiver is coupled to the first and second analog-to-digital converters 115 to transmit the first and second pluralities of digital amplitude values representing the amplitudes of the left and right arterial pressure waves. For such an embodiment, the first and second pluralities of digital amplitude values are transmitted to a separate computing device, such as a smartphone (which may be insertable into or otherwise coupled to the housing 805C), a tablet computer, a laptop or desktop computer, for example and without limitation. The processor 120, memory 125, wireless transceiver 165, user input/output 190 with display 195, and network interface circuit are then located in such a smartphone, a tablet computer, a laptop or desktop computer, and function as described above.

FIGS. 15A and 15B (collectively referred to as FIG. 15) is a flow chart of a representative method embodiment, and provides a useful summary. The method begins, start step 305, with generation of left and right signals, step 310, typically by corresponding signal generators 105. Left and right analog sensor electrical signals are received, step 315, typically by sensors 110. Any additional pressure, temperature, movement, and/or elevation data is received, step 320, such as through additional temperature and pressure sensors 110, accelerometer 140, and/or barometer 145. The left and right analog sensor electrical signals are sampled and converted to corresponding digital amplitude values indicative of or representing the arterial pressure waves (90_{R} or 90_{L}) during the sampling time interval, step 325, typically by the analog-to-digital converters 115. Using the processor 120, the method then determines whether a complete data set has been acquired for one or more arterial pressure waves (90_{R} or 90_{L}), step 330, and not, returns to step 310 and iterates, repeating steps 310-325, to continue to generate signals, receive analog sensor electrical signals, and sample and generate corresponding digital amplitude values. When a complete data set has been acquired for one or more arterial pressure waves (90_{R} or 90_{L}) in step 330, the processor 120 filters and/or performs a fast (or discrete) Fourier transformation of the corresponding digital amplitude values of the arterial pressure waves (90_{R} or 90_{L}), step 335, typically to filter out noise and any motion artifacts, for example and without limitation. The processor 120 also determines, typically using movement, and/or elevation data, whether there has been any movement or posture changes, step 340. The processor 120, typically using digital signal processing components (of DSP block 180), generally generates or determines first mathematical derivatives and possibly also second mathematical derivatives of each left and right arterial pressure waves (90_{R} or 90_{L}), step 345. Using the first and second mathematical derivatives, the processor 120, typically using digital signal processing components (of DSP block 180), generally determines corresponding features, such as corresponding (left and right) foots 80 and/or systolic peaks of 50, as described above, of each left and right arterial pressure waves (90_{R} or 90_{L}), step 350. Using these determined features, the processor 120 then determines the differential pulse arrival time, step 355.

The processor 120 retrieves the calibration data from memory 125, step 360. Using the calibration data, the processor 120 maps or transforms the measured or determined DPAT to the individual's systolic and diastolic blood pressure values, step 365, and determines heart rate and other vital signs, such as stroke volume, as described above, step 370. The processor 120 then outputs the individual's systolic and diastolic blood pressure values, heart rate and other vital signs, step 375, for display to the individual, typically via the user input and output device 190, such as for display to the individual on a touch screen display 195. When the blood pressure determination process is complete, step 380, such as for periodic monitoring, the method may end, return step 385. When the blood pressure determination process is not complete in step 380, such as for ongoing ambulatory monitoring, the method will iterate, returning to step 310.

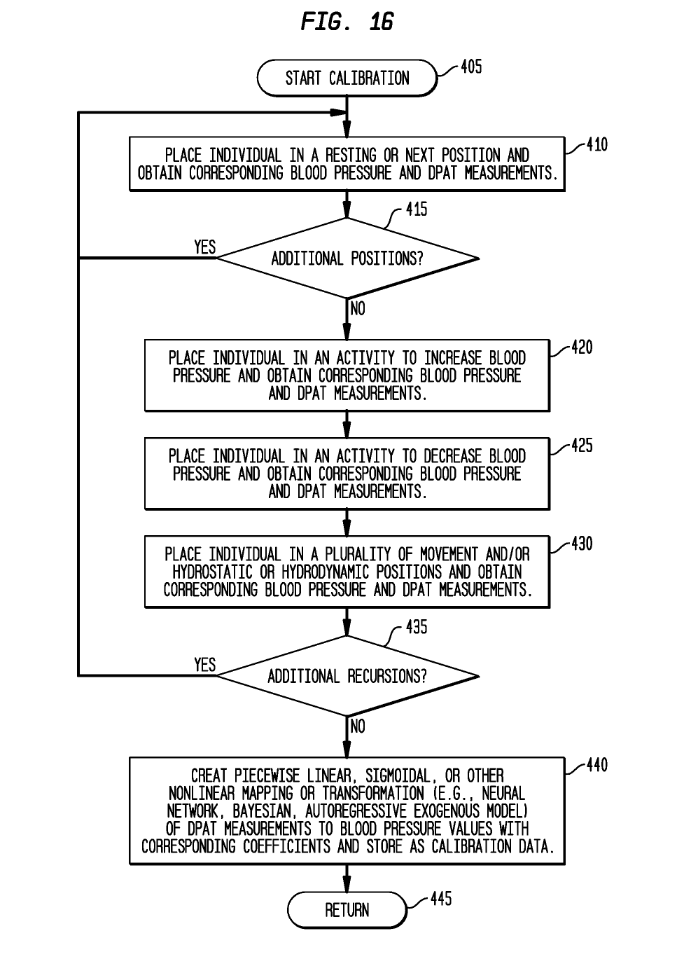

FIG. 16 is a flow chart of a representative method embodiment for the calibration of the representative apparatus and system embodiments for the determination of systolic and diastolic blood pressure values, heart rate and other vital signs. When the system 200, 400, 600 or 700 has not already been calibrated for the individual, a calibration process begins, step 405. For the calibration process, the individual will be placed into a plurality of different positions and engage in a plurality of different activities, during which the individual's systolic and diastolic blood pressure values are obtained independently, such as through a cuff-based system (e.g., using a sphygmomanometer and a stethoscope), and the individual's differential pulse arrival times are determined using the representative apparatus and system embodiments, by performing steps 310 through 355 described above with reference to FIG. 15.

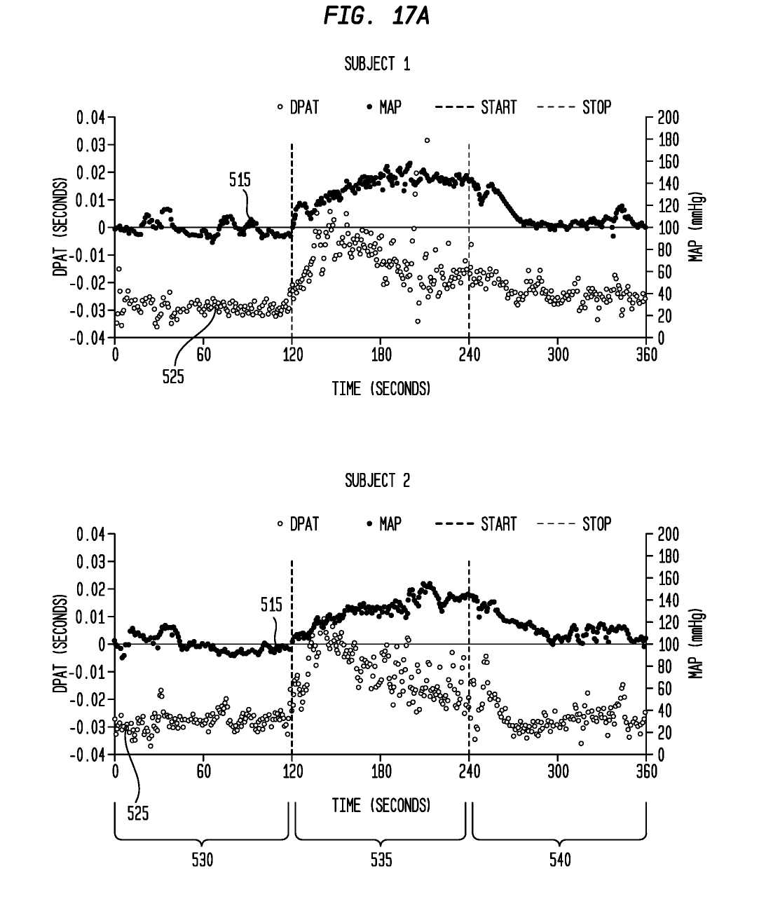

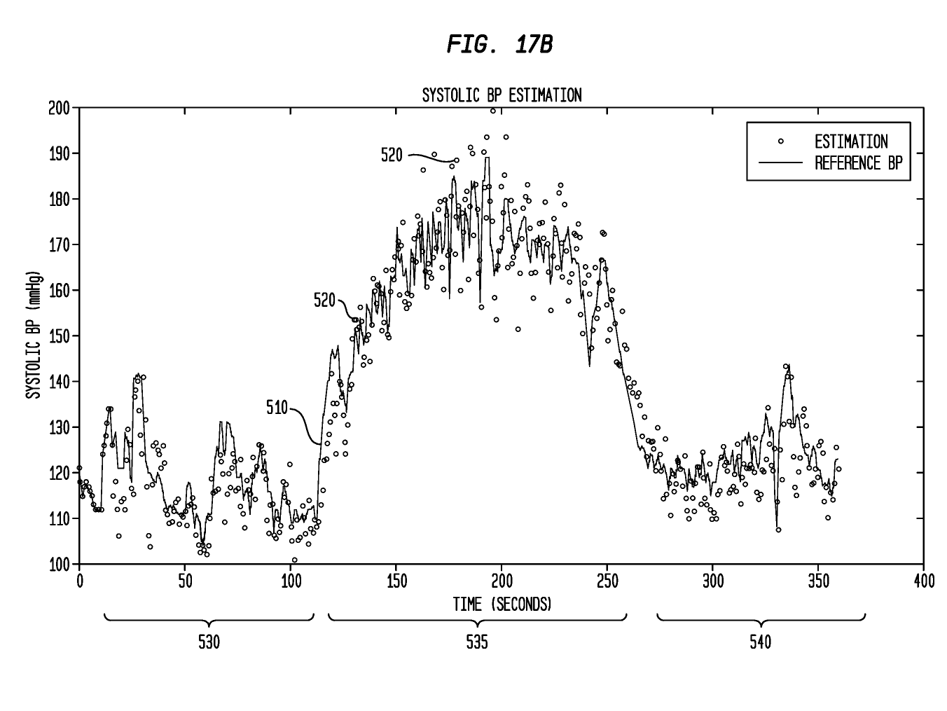

To start the calibration process, step 410, the individual is placed into a resting position, such as sitting, DPAT measurements or determinations are made (performing steps 310 through 355), and corresponding blood pressure values are independently obtained or determined. When there are additional positions for use in calibration, such as having the individual stand or lie down, step 415, this process is repeated, returning to step 410 for each additional position. The individual is then placed into an activity, event or condition, such as performing exercise or a cold pressor test is applied to the individual, which will tend to increase BP, and DPAT measurements or determinations are made (performing steps 310 through 355), and corresponding blood pressure values are independently obtained or determined, step 420. The individual is then placed into an activity, event or condition, such as performing a Valsalva or orthostatic maneuver, which will tend to decrease BP, and DPAT measurements or determinations are made (performing steps 310 through 355), and corresponding blood pressure values are independently obtained or determined, step 425. The individual is then placed into a plurality of different movement and/or hydrostatic or hydrodynamic positions, such as raising and lower arms (when the DPAT measurements, for example, are being made at the left and right wrists, hands, or fingers) which will tend to change the hydrostatics and/or hydrodynamics that may affect the DPAT measurements, and DPAT measurements or determinations are made (performing steps 310 through 355), and corresponding blood pressure values are independently obtained or determined, step 430. This calibration process may then be repeated for additional recursions, step 435. When any additional recursions have been performed, the obtained DPAT measurements or determinations are calibrated to the independently obtained BP values by creating or determining a piecewise-linear mapping of the DPAT measurements or determinations to the independently obtained BP values, or a sigmoidal mapping of the DPAT measurements or determinations to the independently obtained BP values, or a nonlinear, neural network time series analysis using an autoregressive exogenous model, all with corresponding coefficients, and stored as calibration data, step 440, and the calibration process may end, return step 445. Several nonlinear, neural network time series mappings, with an overlay of piecewise-linear or a sigmoidal mappings, of the DPAT measurements or determinations to the independently obtained BP values are illustrated and discussed below with reference to FIGS. 17-21.

By way of background, blood pressure is the force exerted by blood on the vessel wall. The difference between the maximum (systolic) and minimum (diastolic) pressures create a gradient responsible for moving blood throughout the system. The average blood pressure of the physiologic system is defined as the mean arterial pressure (“MAP”). MAP is dictated by total peripheral resistance and cardiac output. Vascular resistance refers to the resistance of the arteries to blood flow such that arterial constriction increases resistance and dilation decreases resistance. The arterial vessel functions as both a conduit for blood and an autonomous regulator of blood pressure by dilating and constricting to modulate resistance. Vessel compliance is the ability of the wall to expand or contract in response to changes in blood pressure and is a function of vessel size and elasticity as follows:

$$C ( P ) = \frac{2 π {r}^{3}}{( E \cdot h )} , where E ( P ) = {E}_{0} {e}^{∝ P} \qquad ( 1 )$$

where elasticity E is recognized to be dependent on arterial pressure P, and where r, E₀, h and ∝ are subject-specific parameters. The mean radial artery diameter, r, may be estimated to be 2.2+/−0.4 mm; the modulus of elasticity, E₀, for a 2 mm diameter artery may be estimated to be 1.88×10⁵ Pa; the thickness of the artery, h, is on average 0.324 mm; and the ∝ coefficient may be estimated to be 0.016.

With hypertension, the velocity of the pulse wave generated by myocardium contraction increases in vessels with reduced compliance and dispensability. The Bramwell-Hill and Mons-Korteweg equations demonstrate the relationship between pulse wave velocity (“PWV”) and vessel elasticity. Specifically, they demonstrate vessel wall elasticity as a function of the elastic modulus and arterial iterance per length L (i.e. pressure to accelerate blood) as follows:

$$PWV = \frac{1}{\sqrt{L \cdot C ( P )}} = \sqrt{\frac{{hE}_{0} {e}^{{∝}^{p}}}{2 r ρ}} = \frac{D}{PTT} \qquad ( 2 )$$

where PTT is the pulse transit time.

The mathematical relationship from DPAT to BP may be estimated through empirical regression models based on the Moens-Kortweg and Bramwell-Hill equations with an assumed function to relate the vessel compliance to BP. In accordance with the representative embodiments, defining DPAT as PTT₁-PTT₂ (e.g., PTT_{R}-PTT_{L} or vice-versa) in (2), and substituting Equation (1) into Equation (2), provides a nonlinear relationship of BP to DPAT (Equation (3)):

BP=K₁ ln(DPAT)+K₂ (3)

where K₁ and K₂, are subject specific coefficients comprised of vessel elasticity, vessel diameter, vessel thickness and distance difference. Using the model of Equation (3) or one of the other models described below, a calibration curve from DPAT to blood pressure can be constructed, as mentioned above, by measuring DPAT and cuff pressure from a subject at rest and also during interventions that perturb blood pressure (e.g., exercise, a cold pressor test, a Valsalva maneuver, etc., as described below), thereby obtaining multiple pairs of PTT and independent BP values, followed by estimating the parameters for that subject by fitting the model to the series of DPAT and BP paired measurements over time. For example and without limitation, as mentioned above, this may be done using a piecewise linear mapping, a sigmoidal mapping, or a nonlinear, neural network time series analysis using an autoregressive exogenous model.

During the calibration process, in addition to DPAT and BP measurements at rest, the subject individual may perform the following:
A. The Valsalva maneuver involves forced expiration against a fixed pressure (typically a closed glottis) that leads to an increased intra-thoracic and intra-abdominal pressure. The maneuver has four physiologic phases: (Phase 1) systolic blood pressure rises due to increased intra-thoracic pressure forcing venous blood into the heart; (Phase 2) systolic blood pressure slowly returns to baseline due to decreased venous return causing a decrease in cardiac output; (Phase 3) the strain is released followed by an abrupt drop in systolic blood pressure below baseline due to acute decrease in intra-thoracic pressure; and (Phase 4) a secondary rise in systolic BP due to a reflex sympathetic response to the decrease in systolic BP seen in Phase 3.
B. Subjects were then asked to maintain aerobic exercise for 5 minutes to elevate heart rate, increase mean arterial pressure, decrease vessel compliance and increase cardiac output. The pulse pressure between the ascending aorta and the brachial/radial artery is also greatly amplified because of a higher relative increase in peripheral compared to central pressure. Higher peripheral vasomotor tone decreases compliance and leads to a faster pulse wave velocity of reflected waves, which are components of the palpated pulse.
C. The cold pressor test is a measurement of vascular reactivity to an external cold stimulus. Blood pressure reactivity to a cold stimulus has been demonstrated to be a reproducible characteristic that correlates with vascular health. Blood pressure sharply rises as a sympathetic response to exposure to cold. The test has commonly been used to evaluate cardiovascular reactivity to stress in normotensive and hypertensive subjects. The test comprises of the participant immersing their lower extremities into an ice water bath (3-5° C.) to just below the knees for 1 minute intervals.

As mentioned above, the calibration is typically performed recursively, e.g., three times in a representative study. Differential pulse arrival time is defined as the time difference between the pulse arriving at the right radial artery and the left radial artery. Negative DPAT values indicate arrival at the right before the left recording site. Data is reported as AVG±SEM. Statistical analysis was conducted using a one-way analysis of variance with a Tukey test for post-hoc evaluation of groups. In all cases, a value of P<0.05 was considered significant.

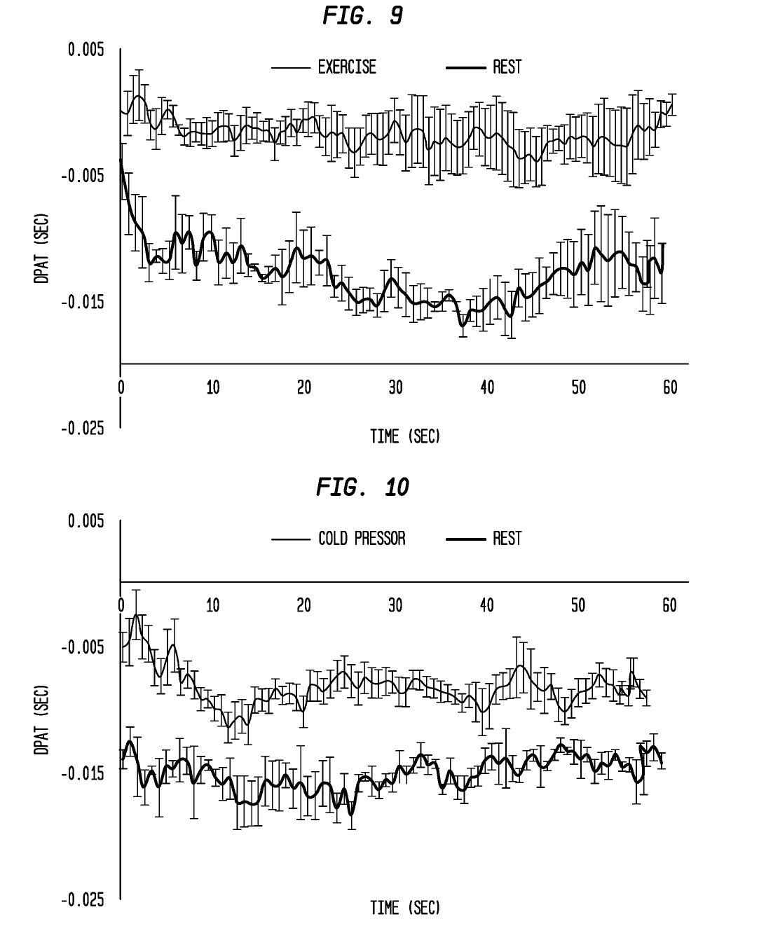

Preliminary results obtained are shown in FIGS. 3-10. The pivotal validation studies demonstrated a strong correlation between differential pulse arrival times and blood pressure in all cases. Further, the studies confirmed an inverse relationship between DPAT and blood pressure in that elevated blood pressures resulted in an increase in pulse waveform velocity and subsequently a decrease in DPAT.

In brief, the average subject resting blood pressure as recorded with a cuff-based home monitor was approximately 130/75 mmHg with a corresponding DPAT value of −0.014±0.000143 seconds. Conversely, exposing the subject to a cold pressor test resulted in a statistically significant increase in blood pressure to approximately 150/80 mmHg. As predicted, the average DPAT value decreased to −0.0087±0.00014 seconds in response to the elevated blood pressure. Similarly, exercise produced a statistically significant rise in blood pressure to 140/90 mmHg with a respective DPAT value of −0.00188±0.000174 seconds. Performance of the Valsalva maneuver provided even greater insight into the relationship between blood pressure and DPAT as the procedure resulted in both an increase and decrease in pressure. As explained above, during the Valsalva maneuver blood pressure initially rises abruptly then consistently drops toward baseline with an overshoot and ultimately a rise again. DPAT tracked these bidirectional changes supporting our hypothesis of an inverse correlation with blood pressure. FIGS. 3-6 illustrated representative waveforms acquired during each procedure of the experiment to demonstrate the phase separation between the waveforms arriving at the right and left radial recording sites. Further, real time beat-to-beat values recorded over a 60 second period are shown in FIGS. 8-10, demonstrating the difference between DPAT values at rest and in response to various environmental stressors.

A calibration and validation study has also been performed using a nonlinear, neural network time series analysis using an autoregressive exogenous model, illustrated in FIGS. 17-21, to detect complex dynamics and dynamic interactions of cardiovascular variables. A nonlinear autoregressive exogenous model (e.g., NARX) can be used to relate the current value of a time series in which one can explain or predict (1) past values of the same series and (2) current and past values of the driving (exogenous) series. For application of the nonlinear autoregressive exogenous model for calibration: (1) an input time-series data string was defined using measured DPAT and heart rate (HR) values, as input (x₁): DPAT (foot-to-foot) (x₁) and HR (x₂); and (2) an output time-series data string was defined using independently measured systolic and diastolic BP values, as output (yₙ): systolic BP or diastolic BP (y₁). All parameters were transformed to zero-mean time-series data, and calibration coefficients were calculated using Equation 4, as a representative NARX model:

$$\hat{y} ( n ) = {c}_{0} + {\sum }_{i = 1}^{Lx} {c}_{i} x ( n - i ) + {\sum }_{i = 1}^{Ly} {d}_{i} y ( n - i ) . \qquad ( 4 )$$

The current value of y(n) (systolic BP or diastolic BP) is then calculated as a prediction from a reference vector formed by the past examples (Lx) of the input parameters series and past examples (Ly) of the output parameter. In a representative embodiment, Lx=5 and Ly=20 were utilized. Coefficients cᵢ and dᵢ may then be estimated through standard least squares estimations, from the K nearest neighbors of the reference vector.

A squared correlation coefficient between the predicted and the actual measurements is obtained as Equation 5:

$${ρ}_{y}^{2} = \frac{{[ {\sum }_{n = L + 1}^{N} y ( n ) \hat{y} ( n ) ]}^{2}}{{\sum }_{n = L + 1}^{N} {y}^{2} ( n ) {\sum }_{n = L + 1}^{N} {\hat{y}}^{2} ( n )} . \qquad ( 5 )$$

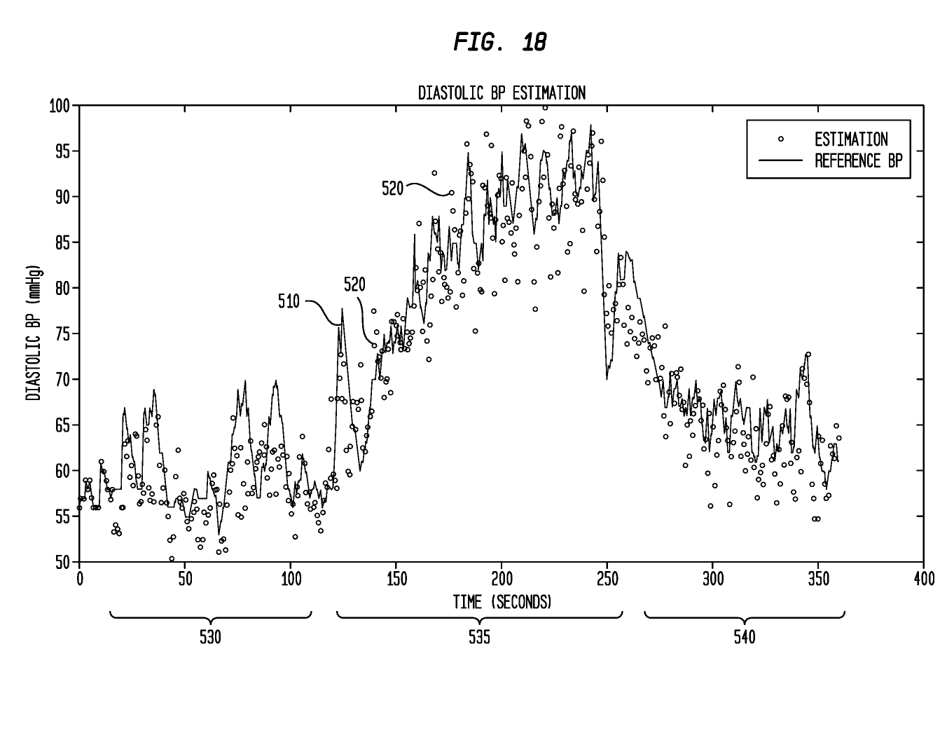

FIGS. 17A and 17B are graphical diagram illustrating, in FIG. 17A, collected DPAT measurements or determinations (represented by the black circles 525, 520) and mean arterial BP measurements (represented by the black dots 515 and line 510) performed using an independent BP device and in FIG. 17B, estimated systolic BP values from collected DPAT measurements or determinations, and systolic BP measurements performed using the independent BP measuring device. FIG. 18 is a graphical diagram illustrating estimated diastolic BP values from collected DPAT measurements or determinations, and diastolic BP measurements performed using the independent BP measuring device.

The independent BP measuring device, for FIGS. 17-22, was a vascular unloading, hemodynamic finger-cuff system (such as a commercially available device from Finapres Medical Systems B.V., Netherlands). FIG. 17A illustrates preliminary data supporting the use of differential pulse arrival time to determine a subject's BP. As illustrated in FIG. 17A, continuous mean arterial pressures (MAP) is shown on the secondary axis in mm Hg and differential pulse arrival times (DPAT) is shown on the primary axis in seconds for 2 individual subjects performing a cold pressor test over the course of 6 minutes. Resting baseline measurements were recorded for 2 minutes (interval 530) prior to the subject placing his/her feet in cold water (40° F.±2° F.) for 2 minutes (interval 535) to elicit a stress response that increased blood pressure (˜+40 mm Hg) before removing their feet from the water and returning to a resting baseline (interval 540). The results confirm that DPAT significantly and reproducibly tracks changes in blood pressure in real time.

As illustrated in FIGS. 17-18, the subject individuals were at rest during a two minute time interval 530, then subject to a cold pressor test during the next two minute time interval 535, followed by a recovery and rest period in the next two minute time interval 540. Blood pressure was measured continuously, every heartbeat, using the independent BP measuring device (Finapres vascular unloading, hemodynamic finger-cuff system, mentioned above), illustrated by the black dots 515 in FIG. 17A and by a line 510 in FIG. 17B, and BP was estimated using concurrently measured or determined DPAT values, represented by the black circles 525, 520 in FIGS. 17A and 17B. The nonlinear autoregressive exogenous model for the calibration of the representative systems 200, 400, 600, 700 proved to be surprisingly robust and accurate, with the BP estimations from the measured or determined DPAT values closely tracking the independently measured (cuff-based) BP values. The systolic BP estimation had a correlation coefficient of 78.67% and a root mean square error (“RMSE”) of 4.76 mmHg, while the diastolic BP estimation had a correlation coefficient 80.32% and an RMSE of 4.03 mmHg. Both of the estimations were done with a 10-beats moving average filter, essentially averaging values over 10 heart beats.

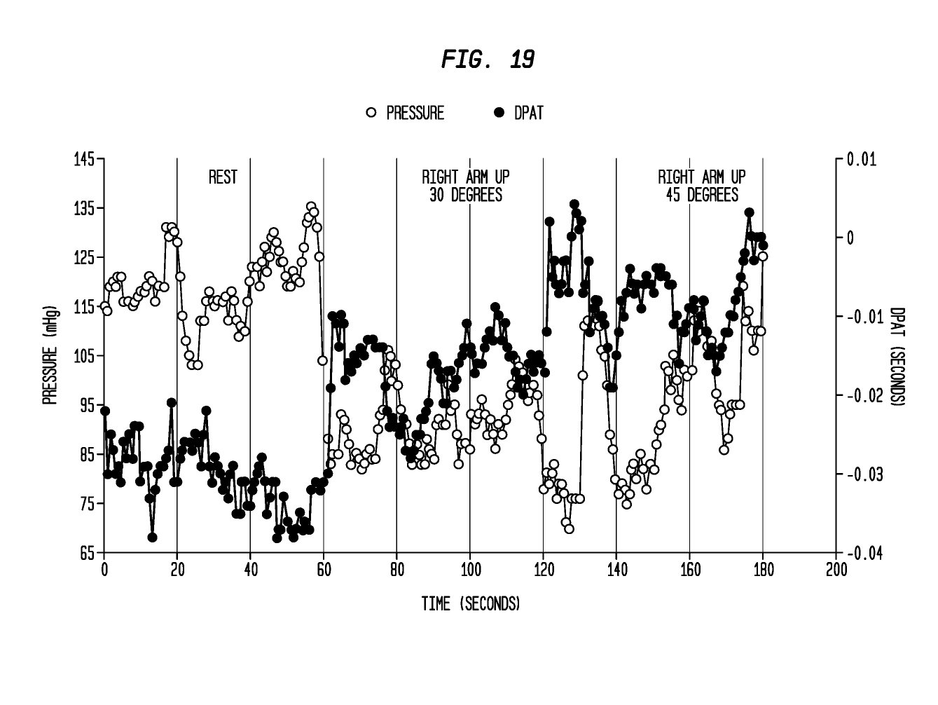

FIG. 19 is a graphical diagram illustrating collected DPAT measurements or determinations (black dots) for systolic BP measurements or determinations, and systolic BP measurements performed using the independent BP measuring device (black circles), for calibration of DPAT measurements or determinations over first and second hydrostatic and/or hydrodynamic movements, conditions or events, as mentioned above with reference to step 430 of FIG. 16. As illustrated in FIG. 19, DPAT measurements or determinations are collected, and systolic BP measurements are performed using the independent BP measuring device and collected, while a subject is at rest (0-60 seconds). Next, DPAT measurements or determinations are collected, and systolic BP measurements are performed using the independent BP measuring device and collected, following the subject raising his or her (right) arm 30 degrees with the left arm at zero degrees as a reference (60-120 seconds) (as a first hydrostatic and/or hydrodynamic movement, condition or event), and again following the subject raising his or her (right) arm further to 45 degrees also with the left arm at zero degrees as a reference (120-180 seconds) (as a second hydrostatic and/or hydrodynamic movement, condition or event). As would be expected, BP will decrease in the raised arm based on hydrostatic forces, while opposition to the pulse wave is increased due to the hydrostatic forces, lowering the pulse velocity in the right arm, resulting in DPAT becoming less negative as the pulse arrival times equalize and the difference in pulse arrival times becomes smaller.

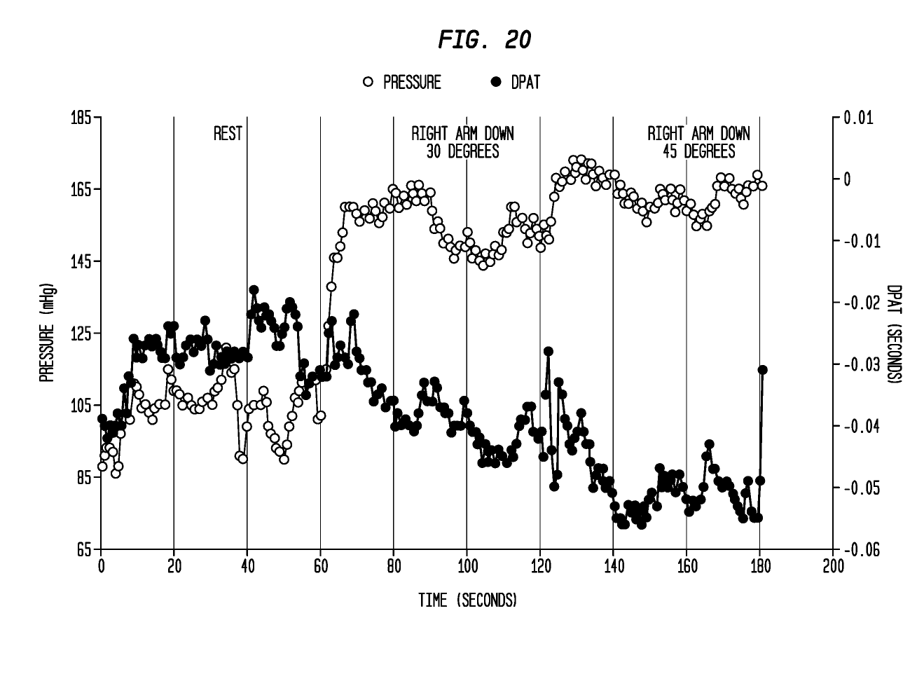

FIG. 20 is a graphical diagram illustrating collected DPAT measurements or determinations for systolic BP measurements or determinations, and systolic BP measurements performed using the independent BP measuring device, for calibration of DPAT measurements or determinations over third and fourth hydrostatic and/or hydrodynamic movements, conditions or events, also as mentioned above with reference to step 430 of FIG. 16. As illustrated in FIG. 20, DPAT measurements or determinations are collected, and systolic BP measurements are performed using the independent BP measuring device and collected, while a subject is at rest (0-60 seconds). Next, DPAT measurements or determinations are collected, and systolic BP measurements are performed using the independent BP measuring device and collected, following the subject lowering his or her (right) arm 30 degrees with the left arm at zero degrees as a reference (60-120 seconds) (as a third hydrostatic and/or hydrodynamic movement, condition or event), and again following the subject lowering his or her (right) arm further to 45 degrees also with the left arm at zero degrees as a reference (120-180 seconds) (as a fourth hydrostatic and/or hydrodynamic movement, condition or event). As would be expected, BP will increase in the lowered arm based on hydrostatic forces, while opposition to the pulse wave is decreased due to the hydrostatic forces, increasing the pulse velocity in the right arm, resulting in DPAT becoming more negative as the difference in pulse arrival times becomes greater.

FIG. 21 is a graphical diagram of FIGS. 19 and 20 illustrating collected DPAT measurements or determinations for systolic BP measurements or determinations, and systolic BP measurements performed using the independent BP measuring device, for calibration of DPAT measurements or determinations over first, second, third and fourth hydrostatic and/or hydrodynamic movements, conditions or events, using a piece-wise linear calibration mapping. As illustrated in FIG. 21, DPAT measurements or determinations may be mapped to absolute, independently determined BP values in a piece-wise linear manner, using piece-wise linear curve 575 (dashed line) for DPAT measurements or determinations and piece-wise linear curve 585 (solid line) for independent BP measurements. For example and without limitation, inflection points may be identified (550, 595, 580, and 635) for the DPAT measurements or determinations and inflection points may be identified (605, 615, 625, and 570) for the BP measurements. In between the inflection points, such as for ranges of DPAT measurements or determinations, corresponding coefficients can be created which can then be utilized to transform DPAT measurements or determinations into corresponding absolute BP values for that range of DPAT values. Stated another way, one or more coefficients can be created in this calibration process which are then utilized to map a range of values of the DPAT measurements or determinations to a corresponding range of BP values. Each of these DPAT ranges mapped to corresponding BP ranges will generally generate corresponding coefficients which can then be utilized to transform any given DPAT measurement or determination within a given range into an absolute BP value for a corresponding BP range, and potentially using interpolated values as well.

FIG. 22 is a graphical diagram of FIGS. 19 and 20 illustrating collected DPAT measurements or determinations for systolic BP measurements or determinations, and systolic BP measurements performed using the independent BP measuring device, for calibration of DPAT measurements or determinations over first, second, third and fourth hydrostatic and/or hydrodynamic movements, conditions or events, using a nonlinear, sigmoidal calibration mapping. As illustrated in FIG. 22, DPAT measurements or determinations may be mapped to absolute, independently determined BP values in a sigmoidal manner, using sigmoidal curve 730 (dashed line) for DPAT measurements or determinations and sigmoidal curve 735 (solid line) for independent BP measurements, as described above for the piece-wise linear curves. For example and without limitation, the corresponding values on the curves 730, 735 for any given regions may be mapped to each other. One or more coefficients can be created in this calibration process using the sigmoidal curves which are then utilized to map a range of values of the DPAT measurements or determinations to a corresponding range of BP values, as described above. Each of these DPAT ranges mapped to corresponding BP ranges on the sigmoidal curves will generally generate corresponding coefficients which can then be utilized to transform any given DPAT measurement or determination within a given range into an absolute BP value for a corresponding BP range, and also potentially using interpolated values as well.

Other calibration methods are also within the scope of the present disclosure, including a recursive Bayesian network mapping and an artificial neural network mapping, for example and without limitation. To achieve a recursive Bayesian network mapping calibration, estimation of BP is being updated each time when a new measurement arrives. Stated another way, a Bayesian calibration provides for modification of a priori probabilities of a DPAT measurement or determination mapping to a given BP based on a posteriori results of the independently measured BP. In other words, an a priori density function at a different state-space (a mathematical model of a physical system as a set of input, output, and state variables) is updated continuously, such as given by Equation 6:

p(xₖ|z_{1:k-1})→p(xₖ|z_{1:k}) (6)

with forward prediction then given by Equation 7:

p(xₖ₋₁|z_{1:k-1})→p(xₖ|z_{1:k-1}) (7).

In this case, the density function is a probability function that estimates DPAT to BP, e.g., a −0.015 seconds DPAT measurement may translate to 92% chance of a BP of 120/80 mm Hg.

Similarly, an artificial neural network mapping will utilize a set of neuron nodes that helps estimate or approximate functions in a reinforcing manner, in which paths between nodes (as probabilities) are strengthened every time a measurement traverses that path. Similar to the recursive Bayesian network, the strengthened connection is analogous to updating an a priori probability density function.

It should also be noted that any of the various calibration calculations and determinations may be made by a separate computing device which receives the corresponding digital amplitude values of the arterial pressure waves (90_{R} or 90_{L}) (from any of the apparatus and/or system embodiments 100, 200, 300, 400, 500, 600, 700) and the BP measurements performed using the independent BP measuring device. The resulting or determined calibration data may then be transmitted or otherwise transferred to the apparatus and/or system embodiments 100, 200, 300, 400, 500, 600, 700, and used as described above.

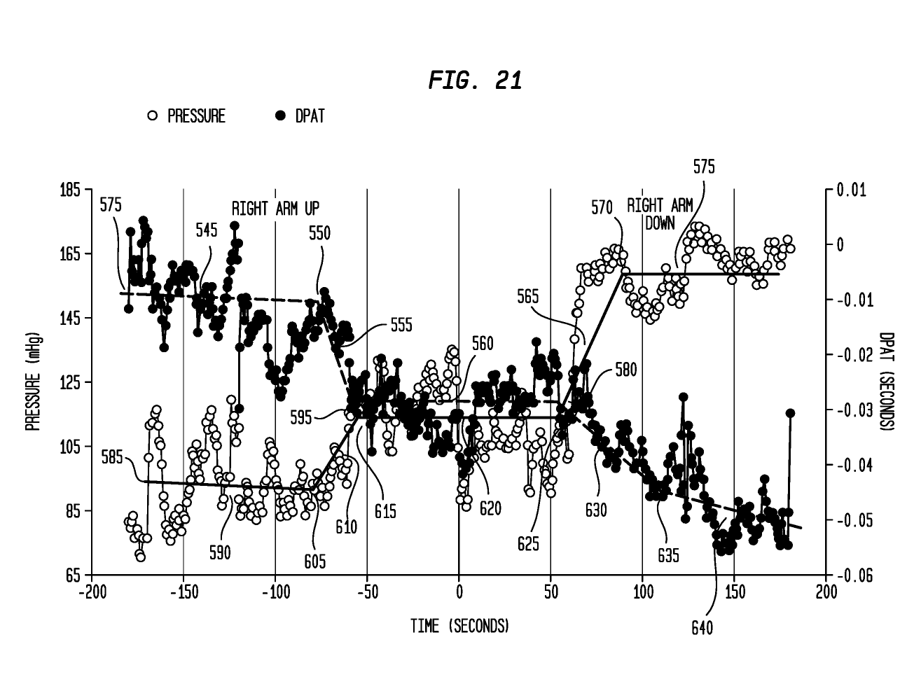

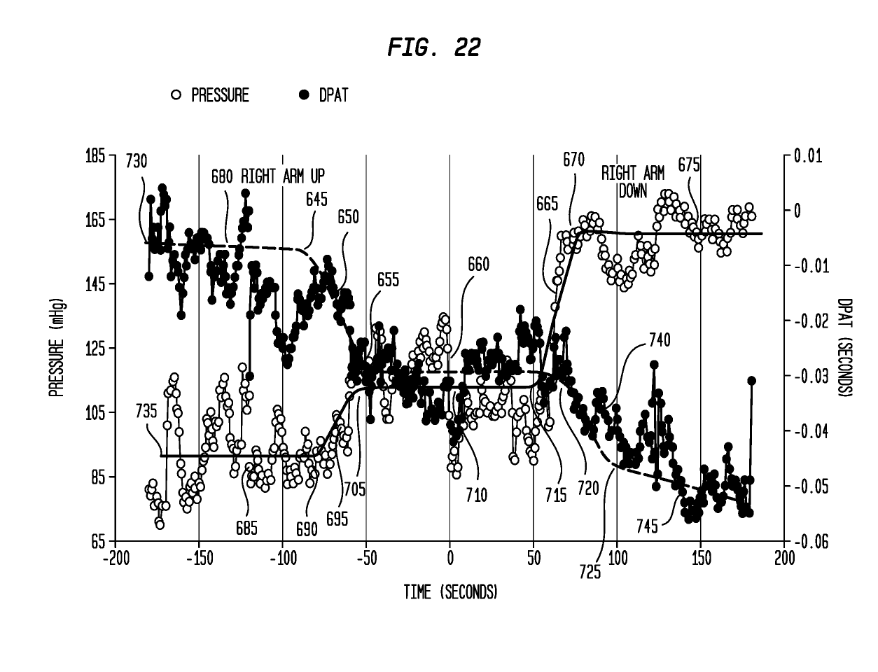

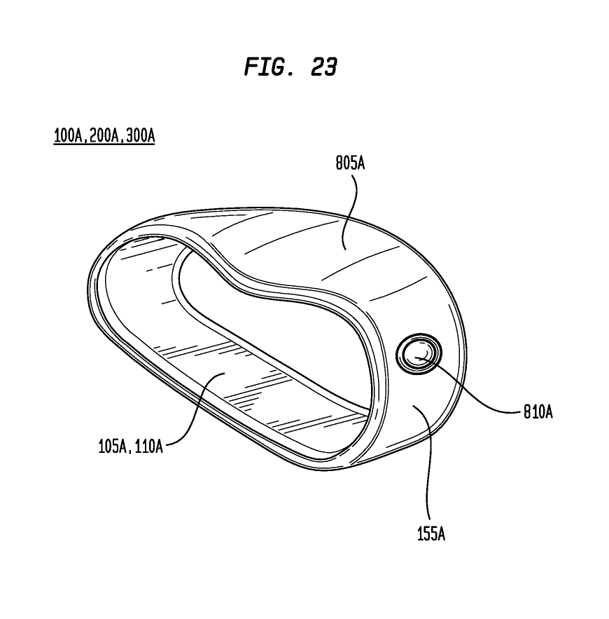

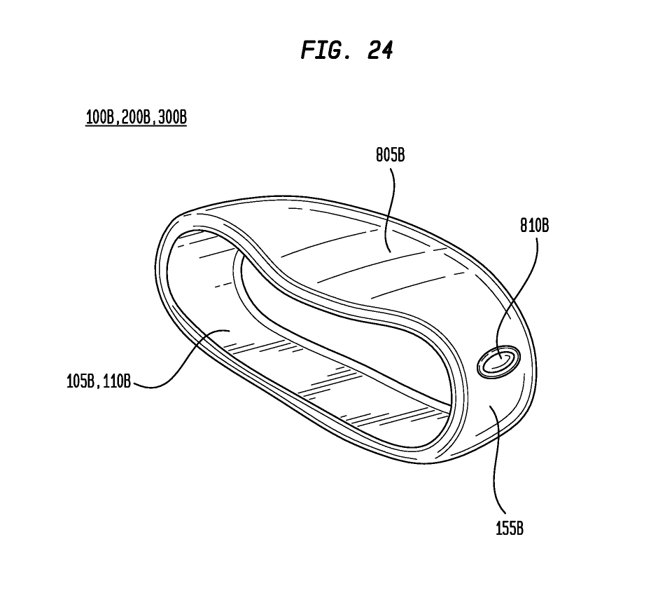

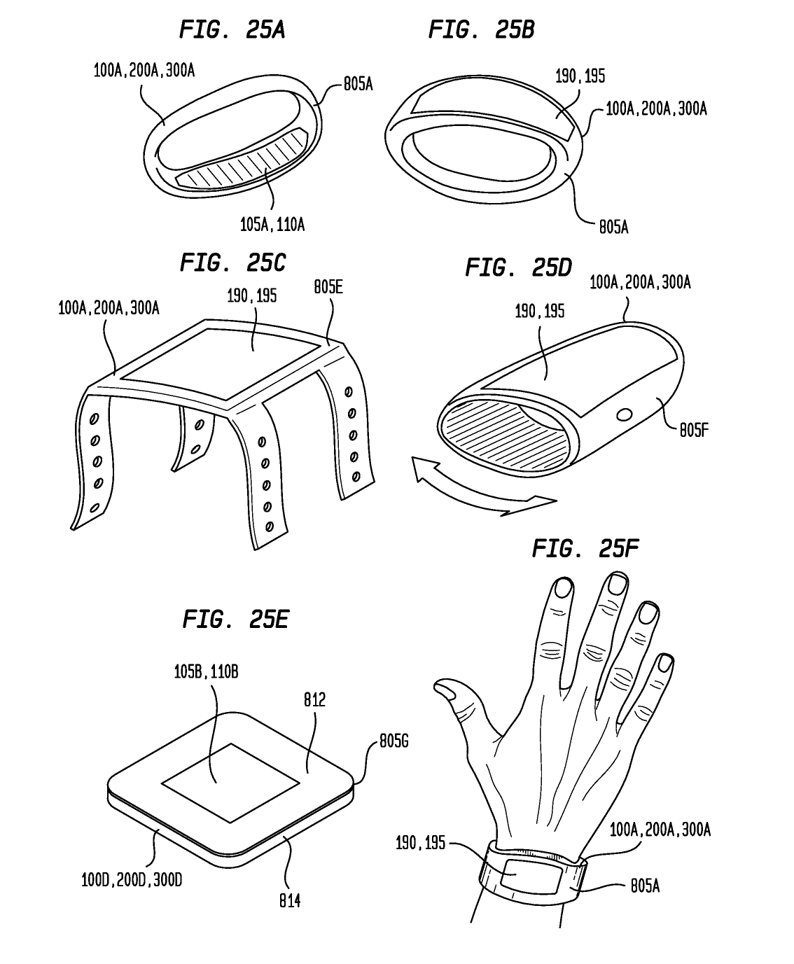

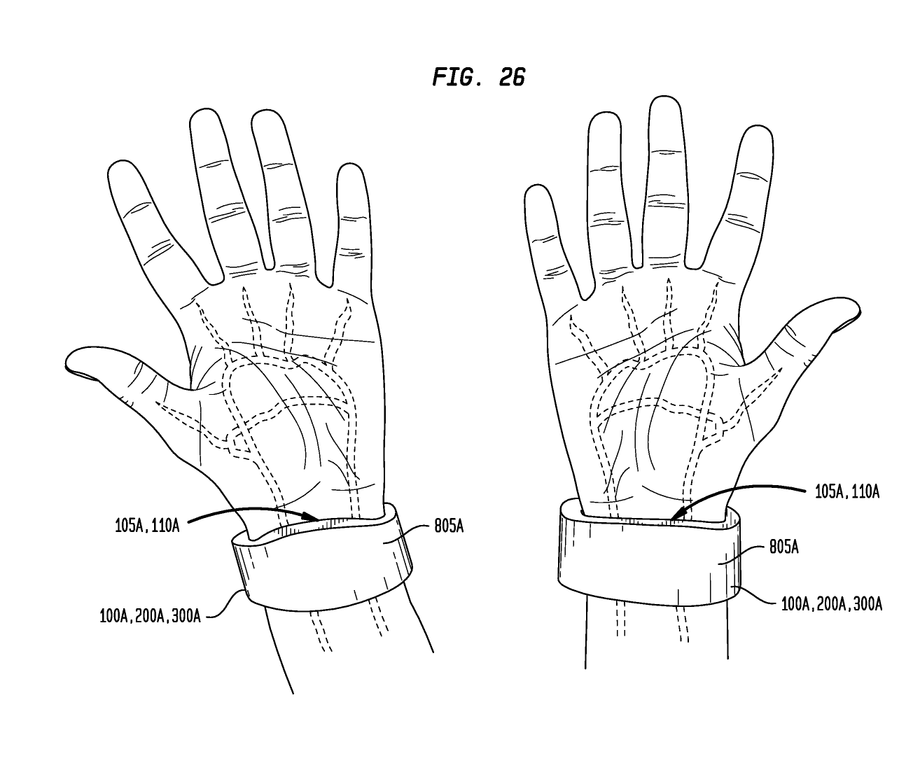

FIG. 23 is an isometric view diagram illustrating representative first, second and/or third apparatus embodiments with a wearable wristband attachment. FIG. 24 is an isometric view diagram illustrating representative first, second and/or third apparatus embodiments with a wearable ring attachment. FIGS. 25A, 25B, 25C, 25D, 25E and 25F (collectively referred to as FIG. 25) are isometric view diagrams illustrating representative first, second and/or third apparatus embodiments with, in FIGS. 25A, 25B, 25C, and 25D, a wearable wristband attachment, in FIG. 25E, a wearable adhesive patch attachment, and in FIG. 25F, a representative first, second and/or third apparatus embodiment with a wearable wristband attachment attached around a wrist of a human subject. FIG. 26 is an isometric view diagram illustrating representative first, second and/or third apparatus embodiment with a wearable wristband attachment attached around a wrist of a human subject. As illustrated in FIGS. 23, 25A, 25B, 25C, 25D, 25F, and 26, the representative first, second and/or third apparatus 100, 200, 300 embodiments, illustrated as first, second and/or third apparatus 100A, 200A, 300A embodiments, have a form factor suitable for wearing on a subject individual's wrist. The signal generator 105A and sensors 110A are located for placement on the volar side of a wrist. Generally, two such apparatuses 100A, 200A, 300A would be worn by a subject individual, one on each left and right wrist, as illustrated in FIG. 26. The electronics of the apparatus 100A, 200A, 300A would generally be included within a housing 805A, which may be part of the wristband wearable attachment 155A. Other features may also be included, such as a charge indicator 810A.

As illustrated in FIG. 24, the representative first, second and/or third apparatus 100, 200, 300 embodiments, illustrated as first, second and/or third apparatus 100B, 200B, 300B embodiments, have a form factor suitable for wearing as a ring on a subject individual's finger. The signal generator 105A and sensors 110A are located for placement on the palmar side of a hand. Generally, two such apparatuses 100B, 200B, 300B would also be worn by a subject individual, one on corresponding finger of left and right hands. The electronics of the apparatus 100B, 200B, 300B would generally be included within a housing 805B, which may be part of the ring wearable attachment 155B. Other features may also be included, such as a charge indicator 810B. Due to potential size constraints of a device having a form factor small enough to be wearable as a ring, only an apparatus 100 is utilized as a representative 100B embodiment.

As illustrated in FIG. 25E, the representative first, second and/or third apparatus 100, 200, 300 embodiments, illustrated as first, second and/or third apparatus 100D, 200D, 300D embodiments, have a form factor suitable for wearing as an adhesive, flexible patch 814, having a comprising an adhesive film 812 and a flexible, biocompatible material suitable for suitable for adhering to multiple and/or different locations on a subject's body as known or becomes known in the art, such as the wrist, upper arm, or neck, for example and without limitation. The signal generator 105A and sensors 110A are located for placement, for example, on the subject's skin in any of these locations, on the side of the adhesive patch 814 with the adhesive film 812. Generally, two such apparatuses 100D, 200D, 300D would also be worn by a subject individual, each one on corresponding locations of the subject individual. The electronics of the apparatus 100D, 200D, 300D would generally be included within a housing 805G, which may be part of the adhesive patch 814. Also due to potential size constraints of a device having a form factor small enough to be wearable as an adhesive patch 814 only an apparatus 100 is utilized as a representative 100D embodiment.

Other variations of these apparatus 100A, 200A, 300A, 100B, 200B, 300B and 100D, 200D, 300D embodiments may be readily apparent and are included within the scope of the disclosure, as mentioned above. For example, the various apparatus 100A, 200A, 300A, 100B, 200B, 300B and 100D, 200D, 300D embodiments may be included and/or distributed between and among a wide variety of housings, such as gloves, finger sleeves, bracelets, etc.

Those having skill in the art will recognize that for such apparatus 100A, 200A, 300A, 100B, 200B, 300B and 100D, 200D, 300D embodiments, the first and second central vital signs monitor 150, 250 may be located in any of a plurality of places and devices. For example, first and second central vital signs monitor 150, 250 may be embodied in a user's computing system or device, a tablet computer, or a smartphone, for example and without limitation, not separately illustrated.

The various systems 200, 400, 600, 700 may be utilized in a variety of contexts and with various other devices. For example and without limitation, an apparatus 100 (as a “slave” device) may transfer its digital amplitude values to any of the apparatus 300 and/or to first and second central vital signs monitor 150, 250 embodiments (as “master” devices), such as via a Bluetooth or other wireless communication connection. Following BP measurements or determinations, any of the apparatus 300 and/or to first and second central vital signs monitor 150, 250 embodiments, in turn, may transfer the resulting data to a “smart” device, such as a smartphone or tablet computer, such as via a Bluetooth or other wireless communication connection. Such a “smart” device, in turn, may generate a summary report, which is uploaded to a centrally-located storage device, such as cloud storage, as mentioned above, for clinician review.

FIG. 27 is an isometric view diagram illustrating representative first, second, third and other apparatus 100C, 200C, 300C, 500C embodiments arranged within a housing 805C such as a smartphone or tablet computer case. A smartphone would be typically placed into the housing 805C on side 825 of the housing 805C, typically facing the user. The opposite side of the housing 805C, side 820, would typically face away from the user, and would have two holes, pads or other placement areas 815_{R} and 815_{L}, containing and exposing corresponding right and left signal generators 105C and sensors 110C, for respective placement of corresponding right and left fingertips for acquisition of DPAT data, as described above. As mentioned above, depending upon the selected embodiment, first and second central vital signs monitor 150, 250 may be embodied in a user's computing system or device, such as a tablet computer or a smartphone, for example and without limitation, which may also be held in the housing 805C.

FIGS. 28 and 29 are isometric view diagrams illustrating a representative fourth apparatus 700A embodiment arranged within a housing 805D, as a singular device. A user input/output device 190 such as a display 195 would be typically placed into the housing 805D on side 835 of the housing 805D, typically facing the user. The opposite side of the housing 805D, side 830, would typically face away from the user, and also would have two holes, pads or other placement areas 815_{R} and 815_{L}, containing and exposing corresponding right and left signal generators 105C and sensors 110C, for respective placement of corresponding right and left fingertips for acquisition of DPAT data, as described above. Corresponding BP measurements, heart rate, and other vital signs may then be displayed to the user on user input/output device 190 such as a display 195.

As mentioned above, there are several advantages to the apparatus 100C, 200C, 300C, 500C, 700A embodiments. The user will typically hold these devices at chest or heart height, with both hands, which significantly decreases motion artifacts that may affect DPAT measurements or determinations. This also tends to significantly decrease any noise which might be affecting the system. In addition, this DPAT measurement or determination may occur without interrupting the user, typically as part of his or her regular activities, such as whenever the user may check his or her email on a smartphone or tablet device held in a housing 805C, for example and without limitation.

As used herein, a “processor” 120 or “controller” 160 may be any type of controller or processor, and may be embodied as one or more processor(s) 120 or controller(s) 160, configured, designed, programmed or otherwise adapted to perform the functionality discussed herein. As the term controller or processor is used herein, a processor 120 or controller 160 may include use of a single integrated circuit (“IC”), or may include use of a plurality of integrated circuits or other components connected, arranged or grouped together, such as controllers, microprocessors, digital signal processors (“DSPs”), array processors, graphics or image processors, parallel processors, multiple core processors, custom ICs, application specific integrated circuits (“ASICs”), field programmable gate arrays (“FPGAs”), adaptive computing ICs, associated memory (such as RAM, DRAM and ROM), and other ICs and components, whether analog or digital. As a consequence, as used herein, the term processor (or controller) should be understood to equivalently mean and include a single IC, or arrangement of custom ICs, ASICs, processors, microprocessors, controllers, FPGAs, adaptive computing ICs, or some other grouping of integrated circuits which perform the functions discussed below, with associated memory, such as microprocessor memory or additional RAM, DRAM, SDRAM, SRAM, MRAM, ROM, FLASH, EPROM or E²PROM. A processor 120 or controller 160, with associated memory, may be adapted or configured (via programming, FPGA interconnection, or hard-wiring) to perform the methodology of the invention, as discussed herein. For example, the methodology may be programmed and stored, in a processor 120 or controller 160 with its associated memory (and/or memory 125) and other equivalent components, as a set of program instructions or other code (or equivalent configuration or other program) for subsequent execution when the processor or controller is operative (i.e., powered on and functioning). Equivalently, when the processor 120 or controller 160 may implemented in whole or part as FPGAs, custom ICs and/or ASICs, the FPGAs, custom ICs or ASICs also may be designed, configured and/or hard-wired to implement the methodology of the invention. For example, the processor 120 or controller 160 may be implemented as an arrangement of analog and/or digital circuits, controllers, microprocessors, DSPs and/or ASICs, collectively referred to as a “processor” or “controller”, which are respectively hard-wired, programmed, designed, adapted or configured to implement the methodology of the invention, including possibly in conjunction with a memory 125.

The memory 125, which may include a data repository (or database), may be embodied in any number of forms, including within any computer or other machine-readable data storage medium, memory device or other storage or communication device for storage or communication of information, currently known or which becomes available in the future, including, but not limited to, a memory integrated circuit (“IC”), or memory portion of an integrated circuit (such as the resident memory within a processor 120, controller 160 or processor IC), whether volatile or non-volatile, whether removable or non-removable, including without limitation RAM, FLASH, DRAM, SDRAM, SRAM, MRAM, FeRAM, ROM, EPROM or E²PROM, or any other form of memory device, such as a magnetic hard drive, an optical drive, a magnetic disk or tape drive, a hard disk drive, other machine-readable storage or memory media such as a floppy disk, a CDROM, a CD-RW, digital versatile disk (DVD) or other optical memory, or any other type of memory, storage medium, or data storage apparatus or circuit, which is known or which becomes known, depending upon the selected embodiment. The memory 125 may be adapted to store various look up tables, parameters, coefficients, other information and data, programs or instructions (of the software of the present invention), and other types of tables such as database tables.

As indicated above, the processor 120 or controller 160 is hard-wired or programmed, using software and data structures of the invention, for example, to perform the methodology of the present invention. As a consequence, the system and method of the present invention may be embodied as software which provides such programming or other instructions, such as a set of instructions and/or metadata embodied within a non-transitory computer readable medium, discussed above. In addition, metadata may also be utilized to define the various data structures of a look up table or a database. Such software may be in the form of source or object code, by way of example and without limitation. Source code further may be compiled into some form of instructions or object code (including assembly language instructions or configuration information). The software, source code or metadata of the present invention may be embodied as any type of code, such as C, C++, Matlab, SystemC, LISA, XML, Java, Brew, SQL and its variations (e.g., SQL 99 or proprietary versions of SQL), DB2, Oracle, or any other type of programming language which performs the functionality discussed herein, including various hardware definition or hardware modeling languages (e.g., Verilog, VHDL, RTL) and resulting database files (e.g., GDSII). As a consequence, a “construct”, “program construct”, “software construct” or “software”, as used equivalently herein, means and refers to any programming language, of any kind, with any syntax or signatures, which provides or can be interpreted to provide the associated functionality or methodology specified (when instantiated or loaded into a processor or computer and executed, including the processor 120, 160, for example).

The software, metadata, or other source code of the present invention and any resulting bit file (object code, database, or look up table) may be embodied within any tangible, non-transitory storage medium, such as any of the computer or other machine-readable data storage media, as computer-readable instructions, data structures, program modules or other data, such as discussed above with respect to the memory 125, e.g., a floppy disk, a CDROM, a CD-RW, a DVD, a magnetic hard drive, an optical drive, or any other type of data storage apparatus or medium, as mentioned above.

The network I/O interface circuit(s) 130 are utilized for appropriate connection to a relevant channel, network or bus; for example, the network I/O interface circuit(s) 130 may provide impedance matching, drivers and other functions for a wireline interface, may provide demodulation and analog to digital conversion for a wireless interface, and may provide a physical interface for the processor 120 or controller 160 and/or memory 125 with other devices. In general, the network I/O interface circuit(s) 130 are used to receive and transmit data, depending upon the selected embodiment, such as program instructions, parameters, configuration information, control messages, data and other pertinent information.

The wireless transmitters 135 and/or wireless transceivers 165 also may be implemented as known or may become known in the art, to provide wireless data communication to and/or from any other device, such as wireless or optical communication and using any applicable standard (e.g., any of the IEEE 802.11 standards, Global System for Mobile Communications (GSM), General Packet Radio Service (GPRS), cdmaOne, CDMA2000, Evolution-Data Optimized (EV-DO), Enhanced Data Rates for GSM Evolution (EDGE), Universal Mobile Telecommunications System (UMTS), Digital Enhanced Cordless Telecommunications (DECT), Digital AMPS (IS-136/TDMA), and Integrated Digital Enhanced Network (iDEN), WCDMA, WiFi, 3G, 4G, and LTE standards, for example and without limitation). In addition, the wireless transmitters 135 and/or wireless transceivers 165 may also be configured and/or adapted to receive and/or transmit signals externally to the systems 200, 400, 600 such as RF or infrared signaling, for example, to receive information in real-time for output on a display, also for example and without limitation.

The network I/O interface circuit(s) 130 may be implemented as known or may become known in the art, to provide data communication between the processor 120 or controller 160 and any type of network or external device, such as wireless, optical, or wireline, and using any applicable standard (e.g., one of the various PCI, USB, RJ 45, Ethernet (Fast Ethernet, Gigabit Ethernet, 100Base-TX, 100Base-FX, etc.), IEEE 802.11, WCDMA, WiFi, GSM, GPRS, EDGE, 3G and the other standards and systems mentioned above, for example and without limitation), and may include impedance matching capability, voltage translation for a low voltage processor to interface with a higher voltage control bus, wireline or wireless transceivers, and various switching mechanisms (e.g., transistors) to turn various lines or connectors on or off in response to signaling from the processor 120 or controller 160. In addition, the network I/O interface circuit(s) 130 may also be configured and/or adapted to receive and/or transmit signals externally to the systems 200, 400, 600 such as through hard-wiring or RF or infrared signaling, for example, to receive information in real-time for output on a display, for example. The network I/O interface circuit(s) 130 may provide connection to any type of bus or network structure or medium, using any selected architecture. By way of example and without limitation, such architectures include Industry Standard Architecture (ISA) bus, Enhanced ISA (EISA) bus, Micro Channel Architecture (MCA) bus, Peripheral Component Interconnect (PCI) bus, SAN bus, or any other communication or signaling medium, such as Ethernet, ISDN, T1, satellite, wireless, and so on.

Numerous advantages of the representative embodiments are readily apparent. The representative apparatus, method and/or system embodiments provide for noninvasive, ambulatory blood pressure and other vital sign monitoring. Representative apparatus and/or system embodiments are comparatively unobtrusive, convenient and easy to use for an individual consumer, while nonetheless being comparatively or sufficiently accurate to obtain meaningful results and actionable information, with a comparatively fast BP acquisition time. Representative apparatus and/or system embodiments also may provide improved compliance by being readily integrable into the user's daily activities. Depending on the selected embodiment, such representative apparatus and/or system embodiments are readily portable and/or wearable to provide ubiquitous monitoring all day and/or night, as may be necessary or desirable.

The present disclosure is to be considered as an exemplification of the principles of the invention and is not intended to limit the invention to the specific embodiments illustrated. In this respect, it is to be understood that the invention is not limited in its application to the details of construction and to the arrangements of components set forth above and below, illustrated in the drawings, or as described in the examples. Systems, methods and apparatuses consistent with the present invention are capable of other embodiments and of being practiced and carried out in various ways.

Although the invention has been described with respect to specific embodiments thereof, these embodiments are merely illustrative and not restrictive of the invention. In the description herein, numerous specific details are provided, such as examples of electronic components, electronic and structural connections, materials, and structural variations, to provide a thorough understanding of embodiments of the present invention. One skilled in the relevant art will recognize, however, that an embodiment of the invention can be practiced without one or more of the specific details, or with other apparatus, systems, assemblies, components, materials, parts, etc. In other instances, well-known structures, materials, or operations are not specifically shown or described in detail to avoid obscuring aspects of embodiments of the present invention. In addition, the various Figures are not drawn to scale and should not be regarded as limiting.

Reference throughout this specification to “one embodiment”, “an embodiment”, or a specific “embodiment” means that a particular feature, structure, or characteristic described in connection with the embodiment is included in at least one embodiment of the present invention and not necessarily in all embodiments, and further, are not necessarily referring to the same embodiment. Furthermore, the particular features, structures, or characteristics of any specific embodiment of the present invention may be combined in any suitable manner and in any suitable combination with one or more other embodiments, including the use of selected features without corresponding use of other features. In addition, many modifications may be made to adapt a particular application, situation or material to the essential scope and spirit of the present invention. It is to be understood that other variations and modifications of the embodiments of the present invention described and illustrated herein are possible in light of the teachings herein and are to be considered part of the spirit and scope of the present invention.

It will also be appreciated that one or more of the elements depicted in the Figures can also be implemented in a more separate or integrated manner, or even removed or rendered inoperable in certain cases, as may be useful in accordance with a particular application. Integrally formed combinations of components are also within the scope of the invention, particularly for embodiments in which a separation or combination of discrete components is unclear or indiscernible. In addition, use of the term “coupled” herein, including in its various forms such as “coupling” or “couplable”, means and includes any direct or indirect electrical, structural or magnetic coupling, connection or attachment, or adaptation or capability for such a direct or indirect electrical, structural or magnetic coupling, connection or attachment, including integrally formed components and components which are coupled via or through another component.

With respect to signals, we refer herein to parameters that “represent” a given metric or are “representative” of a given metric, where a metric is a measure of a state of at least part of the regulator or its inputs or outputs. A parameter is considered to represent a metric if it is related to the metric directly enough that regulating the parameter will satisfactorily regulate the metric. A parameter may be considered to be an acceptable representation of a metric if it represents a multiple or fraction of the metric.

Furthermore, any signal arrows in the drawings/Figures should be considered only exemplary, and not limiting, unless otherwise specifically noted. Combinations of components of steps will also be considered within the scope of the present invention, particularly where the ability to separate or combine is unclear or foreseeable. The disjunctive term “or”, as used herein and throughout the claims that follow, is generally intended to mean “and/or”, having both conjunctive and disjunctive meanings (and is not confined to an “exclusive or” meaning), unless otherwise indicated. As used in the description herein and throughout the claims that follow, “a”, “an”, and “the” include plural references unless the context clearly dictates otherwise. Also as used in the description herein and throughout the claims that follow, the meaning of “in” includes “in” and “on” unless the context clearly dictates otherwise.

The foregoing description of illustrated embodiments of the present invention, including what is described in the summary or in the abstract, is not intended to be exhaustive or to limit the invention to the precise forms disclosed herein. From the foregoing, it will be observed that numerous variations, modifications and substitutions are intended and may be effected without departing from the spirit and scope of the novel concept of the invention. It is to be understood that no limitation with respect to the specific methods and apparatus illustrated herein is intended or should be inferred. It is, of course, intended to cover by the appended claims all such modifications as fall within the scope of the claims.

1. A method of determining a physiological parameter of a subject human being for monitoring, the subject having a left side and a right side, the method comprising:
generating a left signal and a right signal to corresponding left and right positions on the subject;
receiving left and right analog sensor electrical signals from corresponding left and right positions on the subject;
sampling and converting the left and right analog sensor electrical signals into a plurality of digital amplitude values representing amplitudes of left and right arterial pressure waves;
determining corresponding features of the left and right arterial pressure waves;
using the corresponding determined features, measuring a differential pulse arrival time of the left and right arterial pressure waves; and
using the measured differential pulse arrival time, determining at least one physiological parameter selected from the group consisting of: blood pressure, heart rate, stroke rate, and cardiac output.

2. The method of claim 1, wherein the step of determining at least one physiological parameter further comprises:
using calibration data for the subject, mapping the measured differential pulse arrival time to a corresponding blood pressure determined by the calibration data.

3. The method of claim 2, wherein the mapping is selected from the group consisting of: a nonlinear, sigmoidal mapping; a piece-wise linear mapping; a nonlinear autoregressive exogenous mapping; an artificial neural network mapping; a recursive Bayesian network mapping; and combinations thereof.

4. The method of claim 2, wherein the calibration data comprises a plurality of differential pulse arrival times determined for a corresponding plurality of independently determined blood pressure values.

5-15. (canceled)

16. A system for determining a physiological parameter of a subject human being for monitoring, the subject having a left side and a right side, the system comprising:
a plurality of wearable apparatuses, a first wearable apparatus adapted to be worn on the left side, a second wearable apparatus adapted to be worn on the right side, each wearable apparatus of the plurality of wearable apparatuses comprising:
a signal generator to generate either a left signal or a right signal to corresponding left and right positions on the subject;
a sensor to receive a left or right analog sensor electrical signal from corresponding left and right positions on the subject;
an analog-to-digital converter coupled to the sensor to sample and convert the left and right analog sensor electrical signals into a plurality of digital amplitude values representing amplitudes of left and right arterial pressure waves; and
a wireless transmitter coupled to the analog-to-digital converter, the wireless transmitter to transmit the plurality of digital amplitude values; and
a central vital signs monitor, comprising:
a memory circuit to store calibration data for the subject;
a wireless transceiver to receive the transmitted plurality of digital amplitude values; and
a processor coupled to the wireless transceiver and to the memory, the processor adapted to determine corresponding features of the left and right arterial pressure waves; measure a differential pulse arrival time of the left and right arterial pressure waves using the corresponding determined features; and using the measured differential pulse arrival time and the calibration data, to determine at least one physiological parameter selected from the group consisting of: blood pressure, heart rate, stroke rate, and cardiac output.

17. The system of claim 16, wherein the determined physiological parameter is blood pressure, and wherein the processor is further adapted to determine the blood pressure by mapping the measured differential pulse arrival time to a corresponding blood pressure determined by the calibration data, wherein the mapping is selected from the group consisting of: a nonlinear, sigmoidal mapping; a piece-wise linear mapping; a nonlinear autoregressive exogenous mapping; an artificial neural network mapping; a recursive Bayesian network mapping; and combinations thereof.

18. The system of claim 16, wherein the calibration data comprises a plurality of differential pulse arrival times determined for a corresponding plurality of independently determined blood pressure values.

19. The system of claim 16, wherein the calibration data comprises a plurality of differential pulse arrival times determined for a corresponding plurality of independently determined blood pressure values, a plurality of movements, a plurality of temperatures, and a plurality of sensor pressures.

20. The system of claim 16, wherein the processor is further adapted to generate a plurality of first derivatives of the plurality of digital amplitude values; and to determine a corresponding foot of the left and right arterial pressure waves as the corresponding determined features, using the plurality of first derivatives, the plurality of first derivatives indicating a diastolic minimum before a systolic peak and indicating a maximum rate of increasing change in the pressure wave at a rising edge of the systolic peak.

21. The system of claim 16, wherein the signal generator is an optical signal generator to generate light in a predetermined wavelength band.

22. The system of claim 16, wherein the determined physiological parameter is blood pressure, and wherein each wearable apparatus further comprises:
a temperature sensor to receive temperature data; and
a pressure sensor to receive pressure data;
wherein the processor is further adapted to modify the determined blood pressure based upon the received temperature and pressure data.

23. The system of claim 16, wherein the processor is further adapted to filter the plurality of digital amplitude values.

24. The system of claim 16, wherein the determined physiological parameter is blood pressure, and wherein each wearable apparatus further comprises:
an accelerometer to receive movement data;
wherein the processor is further adapted to modify the determined blood pressure based upon the received movement data.

25. The system of claim 16, wherein either the central vital signs monitor or one of the wearable apparatus further comprises:
a visual display device to display the determined physiological parameter value and other vital sign information to the user.

26. The system of claim 16, wherein the wireless transceiver is further adapted to transmit the determined physiological parameter value and other vital sign information to a central location.

27. The system of claim 16, wherein the processor is further adapted to store the determined physiological parameter value and other vital sign information in the memory circuit.

28. The system of claim 16, wherein at least one of the wearable apparatus further comprises a wearable attachment selected from the group consisting of: an adhesive patch, a wristband, a finger ring, a finger sleeve, a finger clip, a glove, an ear clip, and a bracelet.

29. The system of claim 16, wherein the central vital signs monitor is embodied in a separate computing device.

30-52. (canceled)

53. An apparatus for determining a physiological parameter of a subject human being for monitoring, the subject having a left side and a right side, the apparatus utilized in conjunction with a computing device, the apparatus comprising:
a housing having a first, left finger placement location and a second, right finger placement location;
a first signal generator arranged within the housing at the first finger placement location to generate a left signal to a left finger of the subject;
a second signal generator arranged within the housing at the second finger placement location to generate a right signal to a right finger of the subject;
a first sensor arranged within the housing at the first finger placement location to receive a left analog sensor electrical signal from the left finger of the subject;
a second sensor arranged within the housing at the second finger placement location to receive a right analog sensor electrical signal from a right finger of the subject;
a first analog-to-digital converter arranged within the housing and coupled to the first sensor to sample and convert the left analog sensor electrical signals into a first plurality of digital amplitude values representing amplitudes of a left arterial pressure wave;
a second analog-to-digital converter arranged within the housing and coupled to the second sensor to sample and convert the right analog sensor electrical signals into a second plurality of digital amplitude values representing amplitudes of a right arterial pressure wave; and
a wireless transmitter coupled to the first and second analog-to-digital converters to transmit the first and second pluralities of digital amplitude values to the computing device.

54. The apparatus of claim 53, wherein the computing device comprises:
a wireless transceiver to receive the first and second pluralities of digital amplitude values;
a memory circuit to store calibration data for the subject; and
a processor coupled to the memory and to the wireless transceiver, the processor adapted to determine corresponding features of the left and right arterial pressure waves; measure a differential pulse arrival time of the left and right arterial pressure waves using the corresponding determined features; and using the measured differential pulse arrival time and the calibration data, to determine at least one physiological parameter selected from the group consisting of: blood pressure, heart rate, stroke rate, and cardiac output.

55. The apparatus of claim 54, wherein the processor is further adapted to determine the blood pressure by mapping the measured differential pulse arrival time to a corresponding blood pressure determined by the calibration data, wherein the mapping is selected from the group consisting of: a nonlinear, sigmoidal mapping; a piece-wise linear mapping; a nonlinear autoregressive exogenous mapping; an artificial neural network mapping; a recursive Bayesian network mapping; and combinations thereof.

56. The apparatus of claim 54, wherein the calibration data comprises a plurality of differential pulse arrival times determined for a corresponding plurality of independently determined blood pressure values.

57. The apparatus of claim 54, wherein the calibration data comprises a plurality of differential pulse arrival times determined for a corresponding plurality of independently determined blood pressure values, a plurality of movements, a plurality of temperatures, and a plurality of sensor pressures.

58. The apparatus of claim 54, wherein the processor is further adapted to generate a plurality of first derivatives of the plurality of digital amplitude values; and to determine a corresponding foot of the left and right arterial pressure waves as the corresponding determined features, using the plurality of first derivatives, the plurality of first derivatives indicating a diastolic minimum before a systolic peak and indicating a maximum rate of increasing change in the pressure wave at a rising edge of the systolic peak.

59. The apparatus of claim 53, wherein each of the first and second signal generators is an optical signal generator to generate light in a predetermined wavelength band.

60. The apparatus of claim 53, further comprising:
a temperature sensor to receive temperature data; and
a pressure sensor to receive pressure data.

61. The apparatus of claim 60, wherein the determined physiological parameter is blood pressure, and wherein the processor is further adapted to modify the determined blood pressure based upon the received temperature data and pressure data.

62. The apparatus of claim 54, further comprising:
a visual display device to display the determined physiological parameter value and other vital sign information to the user.

63. The apparatus of claim 54, wherein the wireless transceiver is further adapted to transmit the determined physiological parameter value and other vital sign information to a central location.

64. The apparatus of claim 54, wherein the processor is further adapted to store the determined physiological parameter value and other vital sign information in the memory circuit.
